(19) United States
(12) Patent Application Publication
(10) Pub. No.: US 2020/0129535 A1
(43) Pub. Date: Apr. 30, 2020

(54) DESMOCOLLIN 1 INHIBITORS FOR THE PREVENTION OR TREATMENT OF ATHEROSCLEROSIS

(71) Applicants: Jacques GENEST, Dorval (CA); Hong Y. CHOI, Westmount (CA); Adel SCHWERTANI, Montreal (CA)

(72) Inventors: Jacques GENEST, Dorval (CA); Hong Y. CHOI, Westmount (CA); Adel SCHWERTANI, Montreal (CA)

(21) Appl. No.: 16/619,789

(22) PCT Filed: Jun. 4, 2018
(86) PCT No.: PCT/CA2018/050669

§ 371 Date: Dec. 5, 2019

Related U.S. Application Data
(60) Provisional application No. 62/515,548, filed on Jun. 6, 2017.

(51) Int. Cl. A61K 31/702; A61K 38/17; C07K 16/28; C12N 15/113; A61K 31/7048; A61K 31/337; A61P 3/06; G01N 33/68

(52) U.S. Cl. CPC A61K 31/702; A61K 38/1709; C07K 16/2896; C12N 15/1138; G01N 2800/323; A61K 31/337; A61P 3/06; G01N 33/6893; A61K 31/7048

(57) ABSTRACT

There are provided compositions and methods for prevention or treatment of atherosclerosis and related disorders. More specifically, there are provided desmocollin 1 inhibitor compounds and pharmaceutical compositions thereof for use in the prevention or treatment of atherosclerosis and related disorders and/or for promotion of HDL biogenesis in a subject.

### RELATED APPLICATIONS

This application claims priority from U.S. Provisional Application No. 62/515,548 filed Jun. 6, 2017, the entire contents of which are hereby incorporated by reference.

### FIELD

The present disclosure provides compositions and methods for prevention or treatment of atherosclerosis. More specifically, the disclosure relates to desmocollin 1 inhibitor compounds, pharmaceutical compositions thereof and their use in the prevention and/or treatment of atherosclerosis and related disorders and/or for promotion of HDL biogenesis.

### BACKGROUND

Atherosclerotic cardiovascular disease (ASCVD) is characterized as a cholesterol deposition-driven chronic inflammatory disease. The maintenance of cholesterol balance in atherosclerotic lesions is crucial for the prevention and treatment of ASCVD. The major mechanism for cholesterol-laden foam cells to reduce cholesterol burden is to generate high-density lipoprotein (HDL) particles: ATP-binding cassette transporter A1 (ABCA1) upregulated in the foam cells creates specialized microdomains in the plasma membrane (PM) to remove excess cellular cholesterol, where an extracellular lipid acceptor apolipoprotein A-I (apoA-I) binds and solubilizes the domains in order to form apoA-I-lipid complexes termed nascent HDL particles. These particles increase in size by taking up more cholesterol via other lipid transporters such as ATP-binding cassette transporter G1 and scavenger receptor class B type I prior to release into the circulation and delivery of cholesterol to the liver for disposal or recycling, known as reverse cholesterol transport. The rate-limiting step of HDL biogenesis is thus the binding of apoA-I to ABCA1-created PM microdomains. However, the structural and molecular basis of the ABCA1-created PM domains has not been fully determined. Moreover, normal apoA-I binding to cells expressing a dysfunctional or dysregulated ABCA1 indicates the presence of ABCA1-independent apoA-I binding sites on the PM.

The association between high-density lipoprotein cholesterol (HDL-C) and protection against atherosclerotic cardiovascular disease (ASCVD) is strong, coherent and observed across populations. HDL biogenesis occurring in the process of removing excess cellular cholesterol is the most cardiovascular-protective action of HDL, but there is no clinically useful therapy that raises HDL biogenesis. Raising HDL-C with current medications (such as fibric acid derivatives, niacin and the CETP inhibitors torcetrapib, dalcetrapib and evacetrapib) has not shown benefits in terms of ASCVD reduction. Other experimental modulators of HDL have, to date, not shown clinical benefits. These include, for example: apolipoprotein A-I (apoA-I) gene modulators (such as RVX 208, RVX 222); apoA-I or apolipoprotein E mimetic small peptides; and injections of human apoA-I (Milano or wild-type). There is therefore an unmet clinical need for therapies aimed at decreasing ASCVD by increasing HDL functionality.

Desmosomes are intercellular junctions formed by direct binding between desmosomal cadherins, namely the desmogleins (DSGs) and desmocollins (DSCs), at the cell-cell interface. DSGs and DSCs are transmembrane molecules that mediate adhesion through their extracellular domains and serve as a scaffold for assembly of desmosomal plaque through their cytoplasmic domains. In humans, DSG isoforms 1-4 and DSC isoforms 1-3 are encoded by separate genes clustered on chromosome 18. In addition, each of the three DSC isoforms exists in two alternatively spliced forms (a and b), differing only in the carboxyl-terminus. While isoform-specific functions remain to be elucidated, genetic or functional defects in all desmosomal cadherins except DSC1 have been linked to human diseases. Dsc1^{-/-} mice show localized defects in the epidermal barrier, although they can assemble desmosomes normally without compensatory upregulation of other DSC isoforms. Dsc1 knock-in mice that express a truncated DSC1 lacking the carboxyl-terminal tail differentiating DSC1a from DSC1b are free of any phenotype observed in Dsc1^{-/-} mice. It has been reported that the carboxyl-terminal sequences contained in the longer DSC1a but not in the shorter DSC1b are necessary for the recruitment of desmoplakin and plakoglobin to assemble desmosomes. These results support the general idea that DSC1 is dispensable for the assembly of desmosomes, and may harbor its essential functional elements in the extracellular and transmembrane domains.

### SUMMARY

It is an object of the present invention to ameliorate at least some of the deficiencies present in the prior art. Embodiments of the present technology have been developed based on the inventors' appreciation that there is a need for improved compositions and methods for prevention and/or treatment of atherosclerosis and related disorders.

There are provided herein compositions and methods for preventing and/or treating atherosclerosis using a novel therapeutic approach that targets the inhibition or reduction of desmocollin 1 (DSC1), including inhibition or reduction of DSC1 expression, DSC1 binding or interaction with apoA-I, and/or DSC1 biological activity. Compositions and methods provided herein may also be used for prevention and/or treatment of high-density lipoprotein (HDL) biogenesis-linked disorders by inhibiting or reducing DSC1 expression, apoA-I-binding, or activity, thereby promoting HDL biogenesis.

Without wishing to be limited by theory, the present disclosure is based, at least in part, on our findings that the desmocollin 1 (DSC1) protein can bind the apoA-I protein and act as a negative regulator of HDL biogenesis. DSC1 expression levels in atherosclerotic lesions are associated with lesion progression. Further, inhibition of apoA-I-DSC1 protein-protein interactions by knocking down DSC1 expression or by using DSC1-blocking antibodies can increase HDL biogenesis. The DSC1 extracellular region comprises five extracellular cadherin (EC) repeats. Among these EC repeats (EC1-5), the EC5 region comprised of eighty amino acid residues (amino acids 459-538 when numbering starts at the amino-terminal amino acid of mature DSC1) is essential for apoA-I-DSC1 interactions. The EC5 region of the DSC1 protein therefore represents a novel target for the development of inhibitors such as small molecules or monoclonal antibodies that can bind to the EC5 and inhibit apoA-1-DSC1 interactions. Such DSC1-targeting compounds, that can promote HDL biogenesis, are also provided herein.

In a first aspect, there are provided methods of preventing or treating an atherosclerosis-related disorder in a subject in need thereof, comprising administering to the subject an effective amount of a desmocollin 1 (DSC1) inhibitor, such that the atherosclerosis-related disorder is prevented or treated. In some embodiments, the DSC1 inhibitor promotes HDL biogenesis in the subject. The DSC1 inhibitor may be, for example, an inhibitor of DSC1 expression; an inhibitor of DSC1 binding to apoA-I protein; and/or an inhibitor of another DSC1 biological activity.

The DSC1 inhibitor is not meant to be particularly limited. For example, the DSC1 inhibitor may be without limitation a low molecular weight compound, an antibody, a peptide, an antisense oligonucleotide, a small interfering RNA, or another agent sufficient to inhibit DSC1 expression or binding to apoA-I, or inhibit another DSC1 biological activity sufficient to increase HDL biogenesis. In some embodiments, the DSC1 inhibitor binds the EC2 repeat (amino acid residues 130-218 of mature DSC1). In some embodiments, the DSC1 inhibitor binds a region including the EC5 repeat of DSC1 (e.g., a region comprising amino residues 442-538 of mature DSC1, which includes the EC5 repeat at residues 459-538 and the region between the EC4 and EC5 repeats at residues 442-458 of mature DSC1). In some embodiments, the DSC1 inhibitor is a peptide comprising a fragment of apoA-I that blocks DSC1 binding to apoA-I. In some embodiments, the DSC1 inhibitor is a low molecular weight compound set forth in Table 2, or a pharmaceutically acceptable salt or biologically active derivative thereof. In an embodiment, the DSC1 inhibitor is acarbose, rutin, or docetaxel, or a pharmaceutically acceptable salt or biologically active derivative thereof. In other embodiments, the DSC1 inhibitor is an anti-DSC antibody (e.g., a monoclonal antibody) specific for DSC1. In yet other embodiments, the DSC1 inhibitor is an antisense oligonucleotide or a small interfering RNA that targets DSC1 mRNA and/or inhibits DSC1 expression.

In some embodiments, the atherosclerosis-related disorder is atherosclerosis, atherosclerotic cardiovascular disease (ASCVD), or another high-density lipoprotein (HDL) biogenesis-linked disease, disorder or condition. In some embodiments, the subject suffers from, has a predisposition for, or is at risk of, HDL deficiency, a lysosomal storage disease, Tangier disease, Niemann-Pick disease type A, Niemann-Pick disease type B, or Niemann-Pick disease type C.

In a second aspect, there are provided methods of preventing or treating a high-density lipoprotein (HDL) biogenesis-linked disease, disorder or condition in a subject in need thereof, comprising administering to the subject an effective amount of a desmocollin 1 (DSC1) inhibitor, such that the high-density lipoprotein (HDL) biogenesis-linked disease, disorder or condition is prevented or treated. In some embodiments, the DSC1 inhibitor promotes HDL biogenesis in the subject. The DSC1 inhibitor may be, for example, an inhibitor of DSC1 expression, an inhibitor of DSC1 binding to apoA-I protein, and/or an inhibitor of another DSC1 biological activity, as described herein. In some embodiments, the high-density lipoprotein (HDL) biogenesis-linked disease, disorder or condition is atherosclerosis, atherosclerotic cardiovascular disease (ASCVD), HDL deficiency, a lysosomal storage disease, Tangier disease, Niemann-Pick disease type A, Niemann-Pick disease type B, or Niemann-Pick disease type C.

In a third aspect, there are provided methods of promoting HDL biogenesis in a subject in need thereof, comprising administering to the subject an effective amount of a desmocollin 1 (DSC1) inhibitor, such that HDL biogenesis is promoted in the subject. The DSC1 inhibitor may be, for example, an inhibitor of DSC1 expression, an inhibitor of DSC1 binding to apoA-I protein, and/or an inhibitor of another DSC1 biological activity, as described herein. In some embodiments, the subject may suffer from an atherosclerosis-related disorder or a high-density lipoprotein (HDL) biogenesis-linked disease, disorder or condition, such as without limitation atherosclerosis, atherosclerotic cardiovascular disease (ASCVD), HDL deficiency, a lysosomal storage disease, Tangier disease, Niemann-Pick disease type A, Niemann-Pick disease type B, or Niemann-Pick disease type C. It should be understood that, in addition to diseases, disorders and compositions described herein, other diseases, disorders and consitions that can be treated or prevented, in whole or in part, by promotion of HDL biogenesis are candidate indications for the DSC1 inhibitor compounds and compositions provided herein.

In a fourth aspect, there are provided methods of inhibiting desmocollin 1 (DSC1) in a subject in need thereof, comprising administering to the subject a DSC1 inhibitor such that DSC1 expression, DSC1 binding to apoA-I protein, or DSC1 biological activity is inhibited in the subject. The DSC1 inhibitor may be without limitation a low molecular weight compound, an antibody, a peptide, an antisense oligonucleotide, a small interfering RNA, or another agent, as described herein. In particular embodiments, DSC1 inhibitors provided herein act to promote HDL biogenesis, and are useful as therapeutic or prophylactic therapy when such promotion is desired. It should be understood that, in addition to diseases, disorders and compositions described herein, other diseases, disorders and consitions that can be treated or prevented, in whole or in part, by inhibition of DSC1 are candidate indications for the DSC1 inhibitor compounds and compositions provided herein.

In a fifth aspect, there are provided pharmaceutical compositions comprising a desmocollin 1 (DSC1) inhibitor as described herein, or a pharmaceutically acceptable salt or biologically active derivative thereof, and a pharmaceutically acceptable diluent, carrier, or excipient. In some embodiments, there are provided pharmaceutical compositions comprising a compound set forth in Table 2, or a pharmaceutically acceptable salt or biologically active derivative thereof, and a pharmaceutically acceptable diluent, carrier, or excipient. In some embodiments, there are provided pharmaceutical compositions comprising an anti-DSC antibody specific for DSC1, e.g., the EC2 or EC5 repeat regions of DSC1, and a pharmaceutically acceptable diluent, carrier, or excipient. In some embodiments, there are provided pharmaceutical compositions comprising an antisense oligonucleotide or a small interfering RNA that targets DSC1 mRNA, and a pharmaceutically acceptable diluent, carrier, or excipient.

In a sixth aspect, there are provided methods for diagnosing an atherosclerosis-related disorder or a high-density lipoprotein (HDL) biogenesis-linked disease, disorder or condition in a subject, comprising: a) obtaining a biological sample from the subject; b) detecting an expression level of DSC1 in the biological sample; and c) diagnosing the subject as having an atherosclerosis-related disorder or a high-density lipoprotein (HDL) biogenesis-linked disease, disorder or condition, or having a predisposition therefor, or being at risk therefor, when the expression level of DSC1 in the biological sample from the subject is higher than the expression level of DSC1 in a control biological sample from a control subject. In some embodiments, the methods further comprise detecting an expression level of an additional biomarker for atherosclerotic disease in the biological sample, and diagnosing the subject as having an atherosclerosis-related disorder or a high-density lipoprotein (HDL) biogenesis-linked disease, disorder or condition or a predisposition therefor, or being at risk therefor, when the expression level of DSC1 in the biological sample from the subject is higher than the expression level of DSC1 in a control biological sample from a control subject and the expression level of the biomarker is higher or lower than the expression level of the biomarker in the control biological sample. Expression level of DSC1 may be detected, for example, using an anti-DSC1 antibody, a nucleic acid specific for DSC1 RNA, and the like. Non-limiting examples of additional biomarkers include inflammatory biomarkers, biomarkers of endothelial cell, platelet and leukocyte damage, activation, and adhesion, and biomarkers of macrophage monocytes. The biological sample may be without limitation a biological fluid such as whole blood, plasma, serum, tears, saliva, mucous, cerebrospinal fluid, or urine, or a biopsy tissue sample.

In some embodiments of methods provided herein, there is further provided the use of the DSC1 inhibitor compounds and compositions described herein in combination with one or more additional agents. The one or more additional agents may have some DSC1-modulating activity and/or they may function through distinct mechanisms of action. Such agents may comprise, without limitation, cholesterol-lowering drugs (e.g., statins, fibrates, inhibitors of proprotein convertase subtilisin/kexin type 9), blood pressure-lowering therapies, anti-inflammatory agents, anti-thrombotic agents, anti-coagulant agents, inhibitors of the renin-angiotensin aldosterone system (RAAS inhibitors), beta-adrenergic blockers, calcium channel blockers, and/or other treatment modalities of a non-pharmacological nature. When combination therapy is used, the DSC1 inhibitor(s) and one additional agent(s) may be in the form of a single composition or multiple compositions, and the treatment modalities can be administered concurrently, sequentially, or through some other regimen. A combination therapy can have an additive or synergistic effect.

In another aspect, there are provided kits for preventing or treating an atherosclerosis-related disorder or a high-density lipoprotein (HDL) biogenesis-linked disease, disorder or condition, for promoting HDL biogenesis, or for inhibiting DSC1 in a subject, the kits comprising one or more DSC1 compound or composition as described herein. Instructions for use or for carrying out the methods described herein may also be included. A kit may further include additional reagents, solvents, buffers, etc., required for carrying out the methods described herein. Kits for diagnosing atherosclerosis or related disorders comprising reagents for detecting DSC1 expression are also provided.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

For a better understanding of the invention and to show more clearly how it may be carried into effect, reference will now be made by way of example to the accompanying drawings, which illustrate aspects and features according to preferred embodiments of the present invention, and in which:

FIGS. 1(A) to 1(E) show isolation, lipid composition, and protein profile of apoA-I-associated plasma membrane (PM) micro-domains. (A): Extracts of primary human skin fibroblasts were separated by a discontinuous sucrose gradient ultracentrifugation. Among 10 fractions collected, the 4th and 8th fractions contained a visible band. (B): Immunoblot analyses of organelle markers in the 10 fractions indicated in (A) are shown. Under our experimental conditions, apoA-I was associated with PM. The 8th fraction was enriched with apoA-I, PM proteins (ABCA1 and Na/K-ATPase) and endoplasmic reticulum (ER) membrane proteins (ACAT1 and Calnexin). Results are representative of three experiments with similar results. (C): Immunoprecipitation (IP) of the 8th fraction using an anti-apoA-I antibody. The isolated apoA-I-containing pellet excluded two PM proteins, ABCA1 and caveolin. Results are representative of three experiments with similar results. (D): Lipid composition of the apoA-I-associated PM microdomains isolated in (C) is shown. Values represent the averages of three experiments. (E): Proteins of the apoA-I-associated PM microdomains were separated on a 12.5% SDS-polyacrylamide gel and visualized by silver staining. Protein profiles from normal human and Tangier disease (TD) skin fibroblasts are similar. Results are representative of three experiments with similar results.

FIGS. 2(A) to 2(B) show specific binding between apoA-I and DSC1. (A): Primary human skin fibroblasts (HSFs) were incubated with 10 μg/ml of apoA-I for 1 h at 4° C. Proteins extracted from the cells were incubated without antibody or with anti-apoA-I or anti-apoB antibody overnight at 4° C. followed by immunoprecipitation (IP) using Dynabeads Protein G. The precipitate (pellet) and the supernatant (sup) were probed by anti-DSC1 immunoblotting. Results are representative of two experiments with similar results. (B): Primary HSFs were incubated with 10 μg/ml of Alexa Fluor 647-conjugated apoA-I for 1 h at 4° C. The cells were fixed and labeled for DSC1 using an anti-DSC1 antibody. Overlapping fluorescent labels appear as orange to yellow in the merged image. These images are representative of twelve randomly captured fields. Scale bar, 20 μm.

FIGS. 3(A) to 3(C) show that DSC1 binds and co-migrates with apoA-I. (A): HEK293 cells overexpressing DSC1b were incubated with 10 μg/ml of apoA-I for 1 h at 37° C. prior to lysis. The lysate was subjected to immunoprecipitation (IP) without antibody or with anti-DSC1 or anti-DSG1 antibody. The precipitated pellet and the supernatant (Sup) were probed by anti-apoA-I immunoblotting. Results are representative of two experiments with similar results. (B and C): HEK293 cells transfected with pDSC1b-GFP constructs were incubated with 10 μg/ml of Alexa Fluor 647-conjugated apoA-I for 30 min at 37° C. After removing unbound apoA-I by washing cells, two-channel time-lapse images of live cells were captured at 30 sec intervals. (B): The first image set in the time-lapse sequence shows that most of red fluorescent apoA-I locations overlap with green fluorescent DSC1b-GFP locations. The overlapping locations appear as orange to yellow in the merged image. These images are representative of twelve randomly captured fields from three experiments. Scale bar, 5 μm. (C): Sequential images captured over a 5 minute period show dynamic movements of apoA-I and DSC1b-GFP. Arrowheads point the same spot at various time points to track down the movement of an apoA-I-DSC1 complex. Scale bar, 5 μm.

FIGS. 4(A) to 4(D) show the effect of DSC1 on apoA-I-mediated cholesterol efflux. (A): HEK293 cells were transfected with pDSC1a, pDSC1b, pABCA1-GFP, or in combinations as indicated. Two days after the transfection, cells were maintained in Dulbecco's modified Eagle's medium (DMEM) containing 1 mg/ml bovine serum albumin (DMEM/BSA) overnight to deplete serum-derived apoA-I. The cells were incubated with DMEM/BSA containing 5 μg/ml apoA-I for 1 h at 37° C. After extensive washing, the cells were lysed to determine the levels of indicated proteins by immunoblotting. Numeric values shown below the apoA-I blot represent the densities of apoA-I bands normalized to tubulin and relative to mock-transfected cells. In parallel, HEK293 cells labeled with 0.2 μCi/ml of [³H]-cholesterol were subjected to the same transfection scheme. Two days after the transfection, cells were incubated with DMEM/BSA containing 5 μg/ml apoA-I for 24 h to measure apoA-I-mediated cholesterol efflux. Results are displayed as a scatter plot with the mean of quadruplicate determinations. One-way ANOVA with Tukey post-hoc correction was performed to compute multiplicity adjusted p-values. (B): Stable HEK293 cell lines expressing short hairpin RNAs targeting DSC1 (shDSC1) or short hairpin RNAs targeting none (shCont) were transfected with mock or pABCA1-GFP constructs as indicated. Two days after the transfection, cells were incubated with DMDM/BSA for 24 h prior to determining DSC1, ABCA1 and tubulin expression levels by immunoblotting. In parallel, apoA-I-mediated cholesterol efflux assay was performed and the results were analyzed as described in (A). (C): Control HEK293 cells (Cont) and CRISPR/Cas9-mediated DSC1-targeted HEK293 cells (CRISPR-DSC1) were transfected with mock or pABCA1-GFP constructs as indicated. Two days after the transfection, cells were incubated with DMDM/BSA for 24 h prior to determining DSC1, ABCA1 and tubulin expression levels by immunoblotting. In parallel, apoA-I-mediated cholesterol efflux assay was performed and the results were analyzed as described in (A). (D): Primary human skin fibroblasts were labeled with 0.2 μCi/ml of [³H]-cholesterol, loaded with 30 μg/ml of unlabeled cholesterol for 24 h, equilibrated for 24 h, incubated without antibody (none) or with 5 μg/ml of anti-DSC1 or anti-DSG1 antibody for 1 h, and incubated with DMEM/BSA containing 5 μg/ml apoA-I for 24 h to measure apoA-I-mediated cholesterol efflux. Results were analyzed as described in (A).

FIGS. 5(A) to 5(B) show the domain structure of DSC1b protein and mutational analysis of the apoA-I binding site. (A): DSC1b comprises five extracellular cadherin repeats (EC1-5), a single-pass transmembrane domain and an intracellular anchor (IA) domain. EC1-5 repeats were progressively deleted to find which EC repeat is responsible for apoA-I binding. GFP was fused to detect protein expression. (B): HEK293 cells were transfected with constructs indicated. Two days after the transfection, cells were maintained in DMEM/BSA overnight to deplete serum-derived apoA-I. The cells were incubated with DMEM/BSA containing 5 μg/ml apoA-I for 1 h at 37° C. After extensive washing, the cells were lysed to determine the levels of indicated proteins by immunoblotting. Numeric values shown below the apoA-I blot represent the densities of apoA-I bands normalized to actin and relative to mock-transfected cells. Results are representative of three experiments with similar results. The results show that the EC2 and EC5 repeats of the DSC1 extracellular region mediate apoA-I-DSC1 interactions.

FIG. 6 shows co-localization of DSC1 and apoA-I in human coronary atherosclerotic legions. Two serial coronary artery sections obtained from patients with coronary atherosclerosis were immunohistochemically stained for either apoA-I or DSC1. Stained proteins at various stages of atherosclerotic lesions appear brown in color. Co-localization of apoA-I and DSC1 staining was observed in intermediate- and advanced-stage lesions. These images are representative of seven specimens studied.

FIG. 7 shows detection of DSC1 in human carotid atherosclerotic plaques. Carotid artery sections obtained from patients with carotid atherosclerosis were immunohistochemically stained for DSC1. The staining appears brown in color. The progression of carotid atherosclerosis from early to advanced stage was associated with increased DSC1 levels in the plaque. These images are representative of seven specimens studied. These results show that the progression of human carotid atherosclerosis is associated with increased DSC1 levels.

FIGS. 8(A) to 8(B) show that DSC1 is expressed in macrophages. (A): Two serial coronary artery sections obtained from patients with coronary atherosclerosis were immunohistochemically stained for either DSC1 or CD68. DSC1-immunoreactivity is largely localized in CD68-positive cells and the red circle in each panel indicates an area displaying a clear co-localization between DSC1 and CD68. These images are representative of seven specimens studied. (B): DSC1 expression levels in human THP-1 monocytes and macrophages were determined by immunoblotting. A non-specifically detected protein band serves as a loading control. Results are representative of three experiments with similar results.

FIG. 9 is a schematic diagram illustrating a model for plasma membrane (PM) microdomains interacting with apoA-I. ABCA1 creates special PM microdomains for apoA-I to bind and solubilize the domain lipids in the process of HDL particle formation, whereas DSC1 binds apoA-I and prevents apoA-I action in the HDL biogenesis to conserve cholesterol in DSC1-containing desmosomes. By reducing DSC1 expression or inhibiting apoA-I-DSC1 interactions, apoA-I becomes more accessible to ABCA1 microdomains, suggesting that apoA-I-DSC1 binding sites can be targeted to raise HDL biogenesis.

FIGS. 10(A) to 10(B) show the domain structure of DSC1b protein and mutational analysis of apoA-I binding site. (A) shows DSC1b protein comprises five extracellular cadherin repeats (EC1-5), a single-pass transmembrane domain and an intracellular anchor (IA) domain. Green fluorescent protein (GFP) was fused to detect protein expression levels. The EC5 was progressively deleted to investigate if a particular region is responsible for apoA-I binding. (B) shows immunoblot analyses of HEK293 cells that were transfected with the constructs indicated. Two days after the transfection, the cells were maintained in Dulbecco's modified Eagle's medium supplemented with 1 mg/ml bovine serum albumin (DMEM/BSA) overnight to deplete serum-derived apoA-I. The cells were incubated with DMEM/BSA containing 5 μg/ml apoA-I for 1 h at 37° C. After extensive washing, the cells were lysed to determine the levels of indicated proteins by immunoblotting.

FIGS. 11(A) to 11(B) show Ball & Stick (A) and ribbon (B) models of the human DSC1 ectodomain. DSC1 crystal structure 5IRY was prepared by the Protein Preparation Wizard in Maestro for using the five extracellular cadherin (EC) repeats in structure-based virtual screening of ligands. Some of the amino acid residues corrected by the Wizard are shown in (A): HIS, histidine; HIE, histidine with hydrogen on the epsilon nitrogen; HIP, histidine with hydrogens on both nitrogens; Flip, flip the terminal amide group of Asn or Gln; Flip HIS, flip the histidine ring; +2 denotes calcium ion.

FIG. 12 shows the two best binding sites in DSC1 calculated by the SiteMap algorithm. The DSC1 extracellular cadherin (EC) repeats 1 and 5 are predicted to have the first- and second-ranked protein binding sites, respectively. Color scheme for the binding sites displayed in rounded rectangular callouts: hydrogen-bond acceptor regions in blue, hydrogen-bond donor regions in red, hydrophobic regions in yellow, binding site points in white, and the binding site surface in grey.

FIG. 13 shows a display of the second highest-scoring binding site identified by SiteMap. Amino acid residues located within a radius of 3 Å from the binding site are labelled. Hydrogen-bond acceptor regions are coloured blue, hydrogen-bond donor regions in red, hydrophobic regions in yellow, and the binding site surface in grey.

FIG. 14 shows receptor grid generation. The outer, purple enclosing box defines the volume in which the grid potentials were calculated. All atoms of a ligand must be located within the purple box. The inner, green center box defines the volume that the center of a ligand explores during the site-point search. Acceptable positions for the center of a ligand must lie within the green box. Amino acid residues that were corrected by the Protein Preparation Wizard and located within the purple box are labelled with their residue numbers. Calcium ions denoted as +2 are not included in the purple box. HIS: histidine; HIE: histidine with hydrogen on the epsilon nitrogen; Flip: flip the terminal amide group of Asn or Gln residue.

FIG. 15 is a schematic diagram showing the work-flow of DSC1 active site structure-based virtual screening of ligands.

FIG. 16 shows the structures of 51 compounds selected by the structure-based virtual screening strategy.

FIGS. 17(A) to 17(C) show rutin (A), acarbose (B) and docetaxel (C) can promote apoA-I-mediated cholesterol efflux. Primary human skin fibroblasts were labelled with 0.2 μCi/ml of [³H]-cholesterol during growth, loaded with 30 μg/ml of unlabeled cholesterol for 24 h, equilibrated for 24 h, and treated with 5 μg/ml of apoA-I for 24 h to determine efflux of cellular cholesterol by apoA-I. The indicated concentrations of rutin, acarbose and docetaxel were added during the equilibration and apoA-I treatment period. Results are expressed as percentage of total (cell plus medium) [³H]-sterol appearing in the medium. Values are the mean+SD of quadruplicate determinations. One-way analysis of variance with Dunnett's post-hoc correction was performed to calculate multiplicity-adjusted P values. *P<0.05; **P<0.001; ***P<0.0001 compared with the group treated with apoA-I alone.

FIGS. 18(A) to 18(C) show predicted binding poses and interaction diagrams of rutin (A), acarbose (B) and docetaxel (C) in the active site of DSC1 EC5.

FIG. 19 shows the chemical structure of docetaxel with the number of carbon atoms in the taxane ring.

### DETAILED DESCRIPTION

The present disclosure relates to desmocollin 1 (DSC1) inhibitors, to compositions comprising the same and their therapeutic uses in the prevention or treatment of atherosclerosis and related disorders.

In particular, there are provided herein compositions and methods for preventing and/or treating atherosclerosis and related disorders using a novel therapeutic approach that targets the inhibition or reduction of desmocollin 1 (DSC1), including inhibition or reduction of DSC1 expression, inhibition of DSC1 binding to apoA-I, and/or inhibition of DSC1 biological activity. Compositions and methods provided herein may also be used for prevention and/or treatment of high-density lipoprotein (HDL) biogenesis-linked disorders by inhibiting or reducing DSC1 expression, binding, or biological activity.

Without wishing to be limited by theory, compositions and methods provided herein are based, at least in part, on the finding that DSC1 can act as a negative regulator of the apoA-I-mediated cholesterol removal pathway. apoA-I is generally atheroprotective by mediating the formation of HDL particles, in the process removing excess cellular cholesterol from atherosclerotic plaques; interactions with DSC1 inhibit this apoA-I-mediated HDL biogenesis and cholesterol removal. Inhibition of DSC1 by, for example, reducing DSC1 expression, blocking apoA-I-DSC1 interactions, or inhibiting other biological activity of DSC1 can therefore be expected to increase HDL biogenesis thereby providing therapeutic or prophylactic benefit to a wide range of diseases, disorders and conditions associated with defective cholesterol homeostasis.

### Definitions

All technical and scientific terms used herein have the same meaning as commonly understood by one of ordinary skill in the art to which the disclosure pertains. For convenience, the meaning of certain terms and phrases used herein are provided below.

As used herein, the singular forms “a”, “an” and “the” include plural references unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

As used herein, the term “about” is used to indicate that a value includes an inherent variation of error for the device or the method being employed to determine the value. The term “about” generally refers to a value that is within the limits of error of experimental measurement or determination. For example, two values which are about 5%, about 10%, about 15%, or about 20% apart from each other, after correcting for standard error, may be considered to be “about the same” or “similar”. In some embodiments, “about” refers to a variation of +20%, +10%, or +5% from the specified value, as appropriate to perform the disclosed methods or to describe the disclosed compositions and methods, as will be understood by the person skilled in the art.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

Chemical structures herein are drawn according to the conventional standards known in the art. Thus, where an atom, such as a carbon atom, as drawn appears to have an unsatisfied valency, then that valency is assumed to be satisfied by a hydrogen atom even though that hydrogen atom is not necessarily explicitly drawn. Hydrogen atoms should be inferred to be part of the compound.

The symbol “-” in general represents a bond between two atoms in the chain. Thus CH₃—O—CH₂—CH(Rᵢ)—CH₃ represents a 2-substituted-i-methoxypropane compound. In addition, the symbol “-” also represents the point of attachment of the substituent to a compound. Thus for example aryl(C₁-C₆)alkyl- indicates an arylalkyl group, such as benzyl, attached to the compound through the alkyl moiety.

Where multiple substituents are indicated as being attached to a structure, it is to be understood that the substituent can be the same or different. Thus for example “Rₘ optionally substituted with 1, 2 or 3 R_{q} groups” indicates that Rₘ is substituted with 1, 2, or 3 R_{q} groups where the R_{q} groups can be the same or different.

It should be understood that “substitution” or “substituted with” includes the implicit proviso that such substitution is in accordance with the permitted valence of the substituted atom and the substituent, and that the substitution results in a stable compound, i.e., a compound which does not spontaneously undergo transformation such as by rearrangement, cyclization, elimination, etc. As used herein, the term “substituted” is meant to include all permissible substituents of organic compounds. In an embodiment, the permissible substituents include acyclic and cyclic, branched and unbranched, carbocyclic and heterocyclic, aromatic and nonaromatic substituents of organic compounds. The permissible substituents can be one or more. The term “substituted”, when used in association with any of the foregoing groups refers to a group substituted at one or more position with substituents such as acyl, amino (including simple amino, mono and dialkylamino, mono and diarylamino, and alkylarylamino), acylamino (including carbamoyl, and ureido), alkylcarbonyloxy, arylcarbonyloxy, alkoxycarbonyloxy, alkoxycarbonyl, carboxy, carboxylate, aminocarbonyl, mono and dialkylaminocarbonyl, cyano, azido, halogen, hydroxyl, nitro, trifluoromethyl, thio, alkylthio, arylthio, alkylthiocarbonyl, thiocarboxylate, lower alkyl, lower alkenyl, lower alkynyl, cycloalkyl, heterocycloalkyl, aryl, heteroaryl, lower alkoxy, aryloxy, aryloxycarbonyloxy, benzyloxy, benzyl, sulfinyl, alkylsulfinyl, sulfonyl, sulfate, sulfonate, sulfonamide, phosphate, phosphonato, phosphinato, oxo, guanidine, imino, formyl and the like. Any of the above substituents can be further substituted if permissible, e.g., if the group contains an alkyl group, an aryl group, or other.

The technology described herein is not meant to be limited to particular methods, reagents, compounds, compositions or biological systems, which can, of course, vary. It should also be understood that terminology used herein is for the purpose of describing particular aspects only, and is not intended to be limiting.

### DSC1 Inhibitor Compounds

There are provided herein DSC1 inhibitor compounds that can inhibit or reduce DSC1 expression, DSC1 binding to apoA-I, or other DSC1 biological activity, as well as compositions and uses thereof for promotion of HDL biogenesis and/or prevention or treatment of atherosclerosis and related disorders, as well as high-density lipoprotein (HDL) biogenesis-linked diseases, disorders or conditions. It should be understood that any agent capable of inhibiting DSC1 expression, DSC1-apoA-I binding, or other biological activity of DSC1 such that HDL biogenesis is increased in a subject, is intended to be encompassed herein. Non-limiting examples of DSC1 inhibitor compounds are given in Table 2 below and in the Examples.

In an embodiment, there is provided a DSC1 inhibitor compound comprising an antibody specific for the DSC1 protein. In one embodiment, the antibody is specific for the EC2 repeat of DSC1. In another embodiment, the antibody is specific for the EC5 repeat of DSC1. In some embodiments, the antibody specific for DSC1 inhibits or blocks apoA-I binding. In one embodiment, the antibody is a monoclonal antibody directed against the EC5 repeat at amino acid residues 459-538 of mature DSC1. In another embodiments, the antibody is a monoclonal antibody specific for amino acid residues 442-538 of mature DSC1. In another embodiment, the antibody is a monoclonal antibody directed against amino acid residues 130-218 of mature DSC1.

In other embodiments, the DSC1 inhibitor comprises a peptide or small apoA-I fragment that can inhibit apoA-I-DSC1 interactions.

In yet other embodiments, the DSC1 inhibitor comprises an antisense oligonucleotide, a small interfering RNA, a microRNA, or another nucleic acid that targets DSC1 RNA. It should be understood that the method of inhibiting DSC1 expression is not meant to be particularly limited and may include transcriptional regulation, post-transcriptional regulation, translational regulation, and the like, as is well-known to those in the art.

In an embodiment, there is provided a DSC1 inhibitor compound selected from the compounds in Table 2, or a pharmaceutically acceptable salt, or a biologically active derivative thereof. In another embodiment, the DSC1 inhibitor compound is docetaxel or a pharmaceutically acceptable salt or biologically active derivative thereof. In yet another embodiment, the DSC1 inhibitor compound is acarbose or a pharmaceutically acceptable salt or biologically active derivative thereof. In still another embodiment, the DSC1 inhibitor compound is rutin or a pharmaceutically acceptable salt or biologically active derivative thereof.

As would be understood by a person of ordinary skill in the art, the recitation of “a compound” is intended to include salts, solvates, oxides, and inclusion complexes of that compound as well as any stereoisomeric form, or a mixture of any such forms of that compound in any ratio. Compounds described herein include, but are not limited to, their optical isomers, racemates, and other mixtures thereof. In those situations, the single enantiomers or diastereomer, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral high-pressure liquid chromatography (HPLC) column. In addition, such compounds include Z- and E-forms (or cis- and trans-forms) of compounds with carbon-carbon double bonds. Where compounds described herein exist in various tautomeric forms, the term “compound” is intended to include all tautomeric forms of the compound. Such compounds also include crystal forms including polymorphs and clathrates. Similarly, the term “salt” is intended to include all tautomeric forms and crystal forms of the compound.

Thus, in accordance with some embodiments of the invention, a compound as described herein, including in the contexts of pharmaceutical compositions and methods of treatment is provided as the salt form. A “pharmaceutically acceptable salt” of a compound means a salt of a compound that is pharmaceutically acceptable. Desirable are salts of a compound that retain or improve the biological effectiveness and properties of the free acids and bases of the parent compound as defined herein or that take advantage of an intrinsically basic, acidic or charged functionality on the molecule and that are not biologically or otherwise undesirable. Examples of pharmaceutically acceptable salts are also described, for example, in Berge et al., “Pharmaceutical Salts”, J. Pharm. Sci. 66, 1-19 (1977). Non-limiting examples of such salts include:

(1) acid addition salts, formed on a basic or positively charged functionality, by the addition of inorganic acids such as hydrochloric acid, hydrobromic acid, hydroiodic acid, sulfuric acid, sulfamic acid, nitric acid, phosphoric acid, carbonate forming agents, and the like; or formed with organic acids such as acetic acid, propionic acid, lactic acid, oxalic, glycolic acid, pivalic acid, t-butylacetic acid, (3-hydroxybutyric acid, valeric acid, hexanoic acid, cyclopentanepropionic acid, pyruvic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, cyclohexylaminosulfonic acid, benzenesulfonic acid, sulfanilic acid, 4-chlorobenzenesulfonic acid, 2-napthalenesulfonic acid, 4-toluenesulfonic acid, camphorsulfonic acid, 3-phenyl propionic acid, lauryl sulphonic acid, lauryl sulfuric acid, oleic acid, palmitic acid, stearic acid, lauric acid, embonic (pamoic) acid, palmoic acid, pantothenic acid, lactobionic acid, alginic acid, galactaric acid, galacturonic acid, gluconic acid, glucoheptonic acid, glutamic acid, naphthoic acid, hydroxynapthoic acid, salicylic acid, ascorbic acid, stearic acid, muconic acid, and the like;

(2) base addition salts, formed when an acidic proton present in the parent compound either is replaced by a metal ion, including, an alkali metal ion (e.g., lithium, sodium, potassium), an alkaline earth ion (e.g., magnesium, calcium, barium), or other metal ions such as aluminum, zinc, iron and the like; or coordinates with an organic base such as ammonia, ethylamine, diethylamine, ethylenediamine, N,N′-dibenzylethyl enediamine, ethanolamine, diethanolamine, triethanolamine, tromethamine, N-methylglucamine, piperazine, chloroprocain, procain, choline, lysine and the like.

Pharmaceutically acceptable salts may be synthesized from a parent compound that contains a basic or acidic moiety, by conventional chemical methods. Generally, such salts are prepared by reacting the free acid or base forms of compounds with a stoichiometric amount of the appropriate base or acid in water or in an organic solvent, or in a mixture of the two. Salts may be prepared in situ, during the final isolation or purification of a compound or by separately reacting a compound in its free acid or base form with the desired corresponding base or acid, and isolating the salt thus formed. The term “pharmaceutically acceptable salts” also include zwitterionic compounds containing a cationic group covalently bonded to an anionic group, as they are “internal salts”. It should be understood that all acid, salt, base, and other ionic and non-ionic forms of compounds described herein are intended to be encompassed. For example, if a compound is shown as an acid herein, the salt forms of the compound are also encompassed. Likewise, if a compound is shown as a salt, the acid and/or basic forms are also encompassed.

The term “derivative” as used herein refers to a substance similar in structure to another compound but differing in some slight structural detail. The terms “biologically active” and “functionally equivalent” are used interchangeably to refer to derivatives that generally retain biological activity or function of the starting compound, sufficient for use in the present compositions and methods. Thus, a “biologically active” or “functionally equivalent” derivative may retain the DSC1-binding properties (specificity, affinity, etc.) or ability to inhibit DSC1 of the starting compound. In some embodiments, “functionally equivalent” generally refers to a derivative of the compound that maintains sufficient DSC1-binding affinity or specificity for use in the present compositions and methods. In some embodiments, “functionally equivalent” generally refers to a derivative of the compound that maintains sufficient inhibition of DSC1, e.g., inhibition of DSC-1-apoA-I binding, inhibition of DSC1 expression, etc., for use in the present compositions and methods. The DSC1-binding properties or DSC1-inhibition properties of a functionally equivalent compound or derivative need not be identical to those of the reference compound so long as they are sufficient for use in the present compositions and methods for preventing or treating atherosclerosis and related disorders and/or for promoting HDL biogenesis.

The term “substantially pure” is used herein to indicate that a component makes up greater than about 50% of the total content of the composition, and typically greater than about 60% of the total content. More typically, “substantially pure” refers to compositions in which at least 75′%, at least 85%), at least 90% or more of the total composition is the component of interest. In some cases, the component of interest will make up greater than about 90%), or greater than about 95%) of the total content of the composition. In some embodiments, DSC1 inhibitor compounds provided herein are substantially pure.

The term “solvate” refers to a physical association of a compound with one or more solvent molecules, whether organic or inorganic. This physical association includes hydrogen bonding. In certain instances, a solvate will be capable of isolation, for example when one or more solvent molecules are incorporated in the crystal lattice of a crystalline solid. “Solvate” encompasses both solution-phase and isolable solvates. Exemplary solvates include, without limitation, hydrates, ethanolates, methanolates, hemiethanolates, and the like.

### Pharmaceutical Compositions and Methods

There are provided herein compositions and methods for the prevention or treatment of atherosclerosis and related disorders in a subject comprising DSC1 inhibitor compounds described herein. Compositions and methods for promoting HDL biogenesis are also provided. Methods provided herein comprise administration of a DSC1 inhibitor compound to a subject in an amount effective to inhibit or reduce DSC1 expression, inhibit DSC1 binding to apoA-I, or inhibit DSC1 biological activity, and/or to promote HDL biogenesis, thereby reducing, eliminating, preventing, or treating atherosclerosis and related disorders.

As used herein, the term “atherosclerosis” refers to a disease of the arteries characterized by the narrowing of arteries due to plaque buildup in the arteries. The term “atherosclerosis-related disorder” refers to atherosclerotic cardiovascular disease (ASCVD) and other such cholesterol deposition-driven chronic inflammatory diseases. Atherosclerosis-related disorders include, without limitation: ASCVD, coronary heart disease, such as myocardial infarction, angina, and coronary artery stenosis; cerebrovascular disease, such as transient ischemic attack, ischemic stroke, and carotid artery stenosis; peripheral artery disease, such as claudication; aortic atherosclerotic disease, such as abdominal aortic aneurysm and descending thoracic aneurysm; hypertension; peripheral vascular disease; coronary artery disease; aortic aneurysm; carotid artery disease; coronary atherosclerosis; heart attack; acute coronary syndromes, and stroke.

As used herein, the term “high-density lipoprotein (HDL) biogenesis-linked disease, disorder or condition” refers to any disease, disorder or condition for which promotion of HDL biogenesis may be beneficial or protective. In general, HDL biogenesis-linked diseases, disorders or conditions are those in which cholesterol levels play a biological, mechanistic, or pathological role, such that removal of excess cholesterol via HDL biogenesis may be beneficial. Such diseases, disorders and conditions are often associated with defective cholesterol homeostasis. Such diseases, disorders and conditions may also be associated with activity of apoA-I and/or DSC1. Non-limiting examples of HDL biogenesis-linked diseases, disorders and conditions include: atherosclerosis and related disoders (e.g., ASCVD, as discussed above); familial HDL deficiency, Niemann-Pick disease type A; Niemann-Pick disease type B; Niemann-Pick disease type C (NPC); lecithin/cholesterol acyl transferase (LCAT) deficiency; sphingomyelinase deficiency; apoA-I deficiency; ABCA1 deficiency; Tangier disease; dyslipidemia; hypertriglyceridemia; cognitive impairment; Alzheimer's disease; HDL deficiency; and lysosomal storage diseases.

As used herein, the term “promotion of HDL biogenesis” refers to increasing HDL biogenesis and/or creating conditions that favor HDL biogenesis, such that more HDL is produced.

In some embodiments, a DSC1 inhibitor may be used to prevent or treat atherosclerosis or an atherosclerosis-related disorder; to inhibit DSC1, e.g., to inhibit or reduce DSC1 expression, to inhibit or reduce DSC1 binding to apoA-I, or to inhibit or reduce biological activity of DSC1; to prevent or treat an HDL biogenesis-linked disease, disorder or condition; and/or to promote HDL biogenesis.

The terms “administration”, “administer” and the like, as they apply to, for example, a subject, cell, tissue, organ, or biological fluid, refer to contact of, for example, an inhibitor of DSC1, a pharmaceutical composition comprising same, or a diagnostic agent to the subject, cell, tissue, organ, or biological fluid. In the context of a cell, administration includes contact (e.g., in vitro or ex vivo) of a reagent to the cell, as well as contact of a reagent to a fluid, where the fluid is in contact with the cell.

The terms “treat”, “treating”, treatment” and the like refer to a course of action (such as administering an inhibitor of DSC1 or a pharmaceutical composition comprising same) initiated after a disease, disorder or condition, or a symptom thereof, has been diagnosed, observed, and the like, so as to eliminate, reduce, suppress, mitigate, or ameliorate, either temporarily or permanently, at least one of the underlying causes of a disease, disorder, or condition afflicting a subject, or at least one of the symptoms associated with a disease, disorder, condition afflicting a subject. Thus, treatment includes inhibiting (e.g., arresting the development or further development of the disease, disorder or condition or clinical symptoms association therewith) an active disease.

The term “in need of treatment” as used herein refers to a judgment made by a physician or other caregiver that a subject requires or will benefit from treatment. This judgment is made based on a variety of factors that are in the realm of the physician's or caregiver's expertise.

The terms “prevent”, “preventing”, “prevention” and the like refer to a course of action (such as administering a DSC1 inhibitor or a pharmaceutical composition comprising same) initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof: generally in the context of a subject predisposed to having a particular disease, disorder or condition. In certain instances, the terms also refer to slowing the progression of the disease, disorder or condition or inhibiting progression thereof to a harmful or otherwise undesired state.

The term “in need of prevention” as used herein refers to a judgment made by a physician or other caregiver that a subject requires or will benefit from preventative care. This judgment is made based on a variety of factors that are in the realm of a physician's or caregiver's expertise.

The terms “therapeutically effective amount” and “effective amount” are used interchangeably herein to refer to the administration of an agent to a subject, either alone or as part of a pharmaceutical composition and either in a single dose or as part of a series of doses, in an amount capable of having any detectable, positive effect on any symptom, aspect, or characteristic of a disease, disorder or condition when administered to the subject. The therapeutically effective amount can be ascertained by measuring relevant physiological effects, and it can be adjusted in connection with the dosing regimen and diagnostic analysis of the subject's condition, and the like. By way of example, measurement of the serum level of a DSC1 inhibitor (or, e.g., a metabolite thereof) at a particular time post-administration may be indicative of whether a therapeutically effective amount has been used. In some embodiments, the terms “therapeutically effective amount” and “effective amount” refer to the amount or dose of a therapeutic agent, such as a compound, upon single or multiple dose administration to a subject, which provides the desired therapeutic, diagnostic, or prognostic effect in the subject. An effective amount can be readily determined by an attending physician or diagnostician using known techniques and by observing results obtained under analogous circumstances. In determining the effective amount or dose of compound administered, a number of factors are considered including, but not limited to: the size, age, and general health of the subject; the specific disease involved; the degree of or involvement or the severity of the disease or condition to be treated; the response of the individual subject; the particular compound administered; the mode of administration; the bioavailability characteristics of the preparation administered; the dose regimen selected; the use of concomitant medication(s); and other relevant considerations.

DSC1 inhibitors described herein are typically combined with a pharmaceutically acceptable carrier or excipient to form a pharmaceutical composition. Pharmaceutically acceptable carriers can include a physiologically acceptable compound that acts to, e.g., stabilize, or increase or decrease the absorption or clearance rate of a pharmaceutical composition. Physiologically acceptable compounds can include, e.g., carbohydrates, such as glucose, sucrose, or dextrans, antioxidants, such as ascorbic acid or glutathione, chelating agents, low molecular weight proteins, compositions that reduce the clearance or hydrolysis of glycopeptides, or excipients or other stabilizers and/or buffers. Other physiologically acceptable compounds include wetting agents, emulsifying agents, dispersing agents or preservatives which are particularly useful for preventing the growth or action of microorganisms. Various preservatives are well known and include, e.g., phenol and ascorbic acid. Detergents can also be used to stabilize or to increase or decrease the absorption of the pharmaceutical composition, including liposomal carriers. Pharmaceutically acceptable carriers and formulations are known to the skilled artisan and are described in detail in the scientific and patent literature, see e.g., the latest edition of Remington's Pharmaceutical Science, Mack Publishing Company, Easton, Pa. (“Remington's”). One skilled in the art would appreciate that the choice of a pharmaceutically acceptable carrier including a physiologically acceptable compound depends, for example, on the route of administration of the composition, and on its particular physio-chemical characteristics.

Compositions may be administered by any suitable means, for example, orally, such as in the form of pills, tablets, capsules, granules or powders; sublingually; buccally; parenterally, such as by subcutaneous, intravenous, intramuscular, intraperitoneal or intrastemal injection or using infusion techniques (e.g., as sterile injectable aqueous or non-aqueous solutions or suspensions); nasally, such as by inhalation spray, aerosol, mist, or nebulizer; topically, such as in the form of a cream, ointment, salve, powder, or gel; transdermally, such as in the form of a patch; transmucosally; or rectally, such as in the form of suppositories. The present compositions may also be administered in a form suitable for immediate release or extended release. Immediate release or extended release may be achieved by the use of suitable pharmaceutical compositions, or, particularly in the case of extended release, by the use of devices such as subcutaneous implants or osmotic pumps.

Pharmaceutical compositions provided herein can be formulated to be compatible with the intended method or route of administration; exemplary routes of administration are set forth herein. Furthermore, the pharmaceutical compositions may be used in combination with other therapeutically active agents or compounds as described herein in order to treat or prevent the DSC1-associated diseases, disorders and conditions as contemplated herein.

Pharmaceutical compositions containing the active ingredient (e.g., a DSC1 inhibitor) may be in a form suitable for oral use, for example, as tablets, capsules, troches, lozenges, aqueous or oily suspensions, dispersible powders or granules, emulsions, hard or soft capsules, or syrups, solutions, microbeads or elixirs. Pharmaceutical compositions intended for oral use may be prepared according to any method known in the art for the manufacture of pharmaceutical compositions, and such compositions may contain one or more agents such as, for example, sweetening agents, flavoring agents, coloring agents and preserving agents in order to provide pharmaceutically acceptable preparations. Tablets, capsules and the like generally contain the active ingredient in admixture with non-toxic pharmaceutically acceptable carriers or excipients which are suitable for the manufacture of tablets. These carriers or excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

Tablets, capsules and the like suitable for oral administration may be uncoated or coated using known techniques to delay disintegration and absorption in the gastrointestinal tract and thereby provide a sustained action. For example, a time-delay material such as glyceryl monostearate or glyceryl distearate may be employed. They may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylenevinyl acetate, methycellulose, carboxymethylcellulose, protamine sulfate, or lactide/glycolide copolymers, polylactide/glycolide copolyrners, or ethylenevinylacetate copolyrners in order to control delivery of an administered composition. For example, the oral agent can be entrapped in microcapsules prepared by coacervation techniques or by interfacial polymerization, using hydroxymethylcellulose or gelatin-microcapsules or poly (methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods for the preparation of the above-mentioned formulations will be apparent to those skilled in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil. Aqueous suspensions contain the active materials in admixture with excipients suitable for the manufacture thereof. Such excipients can be suspending agents, for example sodium carboxymethylcellulose, methykellulose, hydroxy-propylmethyl cellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol rnonooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are known in the art.

Pharmaceutical compositions of the present invention may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

Pharmaceutical compositions typically comprise a therapeutically effective amount of a DSC1 inhibitor compound provided herein and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bi sulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, phamrnceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl)piperazine-N′-(2-ethanesulfonic acid) (HEPES), 2-(N-MoqJholino)ethanesulfonic acid (MES), 2-(N-Morpholino)ethanesulfonic acid sodium salt (MES), 3-(N-Morpholino)propanesulfonic acid (MOPS), and Ntris[Hydroxyrnethyl]methyl-3-arninopropanesulfonic acid (TAPS). After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form.

In some embodiments, the pharmaceutical composition is provided in a single-use container (e.g., a single-use vial, ampoule, syringe, or autoinjector, whereas a multi-use container (e.g., a multi-use vial) is provided in other embodiments.

Formulations can also include carriers to protect the composition against rapid degradation or elimination from the body, such as a controlled release formulation, including liposomes, hydrogels, prodrugs and microencapsulated delivery systems. For example, a time delay material such as glyceryl monostearate or glyceryl stearate alone, or in combination with a wax, may be employed. Any drug delivery apparatus may be used to deliver a DSC1 inhibitor, including implants (e.g., implantable pumps) and catheter systems, slow injection pumps and devices, all of which are well known to the skilled artisan.

Pharmaceutical compositions may also be in the form of a sterile injectable aqueous or oleagenous suspension. This suspension may be formulated according to the known art using those suitable dispersing or wetting agents and suspending agents mentioned herein. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor ELTM (BASF, Parsippany, N.J.) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose any bland fixed oil may be employed, including synthetic mono- or diglycerides. Moreover, fatty acids such as oleic acid, find use in the preparation of injectables. Prolonged absorption of particular injectable formulations can be achieved by including an agent that delays absorption (e.g., aluminum monostearate or gelatin).

DSC1 inhibitor compounds and compositions provided herein may be administered to a subject in any appropriate manner known in the art. Suitable routes of administration include, without limitation: oral, parenteral (e.g., intramuscular, intravenous, subcutaneous (e.g., injection or implant), intraperitoneal, intracisternal, intraarticular, intracerebral (intraparenchymal) and intracerebroventricular), nasal, vaginal, sublingual, intraocular, rectal, topical (e.g., transdermal), buccal and inhalation. Depot injections, which are generally administered subcutaneously or intramuscularly, may also be utilized to release the DSC1 inhibitors disclosed herein over a defined period of time. In certain embodiments, DSC1 inhibitor compounds and compositions are administered orally to a subject in need thereof.

DSC1 inhibitor compounds and compositions provided herein may be administered to a subject in an amount that is dependent upon, for example, the goal of administration (e.g., the degree of resolution desired); the age, weight, sex, and health and physical condition of the subject to which the formulation is being administered; the route of administration; and the nature of the disease, disorder, condition or symptom thereof. The dosing regimen may also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan. In general, dosing parameters dictate that the dosage amount be less than an amount that could be irreversibly toxic to the subject (the maximum tolerated dose (MID)) and not less than an amount required to produce a measurable effect on the subject. Such amounts are determined by, for example, the pharmacokinetic and pharmacodynamic parameters associated with ADME, taking into consideration the route of administration and other factors.

In some embodiments, a DSC1 inhibitor may be administered (e.g., orally) at dosage levels of about 0.01 mg/kg to about 50 mg/kg, or about 1 mg/kg to about 25 mg/kg, of subject body weight per day, one or more times a day, to obtain the desired therapeutic effect. For administration of an oral agent, the compositions can be provided in the form of tablets, capsules and the like containing from 1.0 to 1000 milligrams of the active ingredient, particularly 1, 3, 5, 10, 15, 20, 25, 50, 75, 100, 150, 200, 250, 300, 400, 500, 600, 750, 800, 900, or 1000 milligrams of the active ingredient.

In some embodiments, the dosage of the desired DSC1 inhibitor is contained in a “unit dosage form”. The phrase “unit dosage form” refers to physically discrete units, each unit containing a predetermined amount of the DSC1 inhibitor, either alone or in combination with one or more additional agents, sufficient to produce the desired effect. It will be appreciated that the parameters of a unit dosage form will depend on the particular agent(s) and the effect to be achieved.

It will be understood that the specific dose level and frequency of dosage for any particular subject may be varied and will depend upon a variety of factors including the activity of the specific compound employed, the metabolic stability and length of action of that compound, the species, age, body weight, general health, sex and diet of the subject, the mode and time of administration, rate of excretion and clearance, drug combinations, and severity of the particular condition.

The terms “subject” and “patient” are used interchangeably herein to refer to a human or a non-human animal (e.g., a mammal). A subject may be a vertebrate, such as a mammal, e.g., a human, a non-human primate, a rabbit, a rat, a mouse, a cow, a horse, a goat, or another animal. Animals include all vertebrates, e.g., mammals and non-mammals, such as mice, sheep, dogs, cows, avian species, ducks, geese, pigs, chickens, amphibians, and reptiles. In an embodiment, a subject is a human. In some embodiments, a subject is in need of prevention or treatment for atherosclerosis or a related disorder or condition, or an HDL-biogenesis linked disease, disorder or condition.

In some embodiments, compositions provided herein include one or more additional therapeutic or prophylactic agents for atherosclerosis and related disorders or conditions or HDL-biogenesis linked diseases, disorders or conditions. For example, a composition may contain a second agent for preventing or treating atherosclerosis. Examples of such second agents include, without limitation, statins, anti-platelet medications, beta blocker medications, angiotensin-converting enzyme (ACE) inhibitors, calcium channel blockers, and diuretics.

In alternative embodiments, compositions of the present invention may be employed alone, or in combination with other suitable agents useful in the prevention or treatment of atherosclerosis and related disorders or conditions or HDL-biogenesis linked diseases, disorders or conditions. In some embodiments compositions of the present invention are administered concomitantly with a second composition comprising a second therapeutic or prophylactic agent for atherosclerosis and related disorders or conditions or for HDL-biogenesis linked diseases, disorders or conditions.

### Diagnostics

There are also provided herein methods for diagnosing and monitoring atherosclerosis and related diseases using one or more samples obtained from a subject. The methods comprise detecting an expression level of desmocollin 1 (DSC1) in a biological sample from a subject, and diagnosing the patient as having atherosclerosis or a related disease, or a predisposition or risk therefor, when the expression level of DSC1 in the subject is higher than the normal expression level of DSC1 in a biological sample from a control subject. Diagnostic methods provided herein may also be used to monitor atherosclerotic disease progression and/or to monitor a subject's treatment, response to therapy, etc.

In some embodiments, the methods provided herein further comprise detecting an expression level of one or more additional biomarker for atherosclerotic disease in a biological sample from a subject, and diagnosing the patient as having atherosclerosis or a related disorder, or a predisposition or risk therefor, when the expression level of DSC1 and the one or more additional biomarker in the subject is higher than the normal expression level of DSC1 and the one or more additional biomarker in a biological sample from a control subject. The one or more additional biomarker may be any biomarker known to be associated with atherosclerotic disease, such as an inflammatory biomarker, a biomarker of endothelial cell, platelet and leukocyte damage, activation, and adhesion, a biomarker of macrophages and monocytes, etc. Non-limiting examples of such markers include the proteins RANTES, TIMP 1, MCP-1, MCP-2, MCP-3, MCP-4, eotaxin, IP-10, M-CSF, IL-3, TNFa, Ang-2, IL-5, IL-7, IGF-1, sVCAM, sICAM-1, E-selectin, P-selection, interleukin-6, interleukin-18, C-reactive protein, creatine kinase, LDL, oxLDL, LDL particle size, apolipoprotein B, Lipoprotein(a), troponin I, troponin T, Lp-PLA2, HDL-cholesterol, apolipoprotein A-I, Triglyceride, insulin, BNP, fractalkine, osteopontin, osteoprotegerin, oncostatin-M, Myeloperoxidase, ADMA, PAI-1 (plasminogen activator inhibitor), SAA (circulating amyloid A), t-PA (tissue-type plasminogen activator), sCD40 ligand, fibrinogen, homocysteine, D-dimer, leukocyte count, heart-type fatty acid binding protein, Lipoprotein (a), MMP1, Plasminogen, folate, omega-3 fatty acids, vitamin B6, vitamin D, Leptin, soluble thrombomodulin, PAPPA, MMP9, MMP2, VEGF, PIGF, HGF, vWF, and cystatin C.

Expression levels of DSC1 and other biomarkers may be determined using standard methods known in the art. For example, RNA and/or protein levels may be determined using any capture agent specific for the RNA or protein in question, such as an antibody, fragment, analog, conjugate, or chemical that specifically detects the RNA or protein being measured. A capture agent may be a protein or antibody that specifically binds DSC1, an oligonucleotide that specifically binds to DSC1 RNA, etc. In some embodiments, a capture reagent is coupled to a solid support and/or to a detectable label.

As used herein, “capture agent” refers to a molecule or group of molecules that specifically bind to a specific target molecule or group of target molecules. For example, a capture agent can comprise two or more antibodies each antibody having specificity for a separate target molecule. Capture agents can be any combination of organic or inorganic chemicals, or biomolecules, and all fragments, analogs, homologs, conjugates, and derivatives thereof that can specifically bind a target molecule. The capture agent can comprise a single molecule that can form a complex with multiple targets, for example, a multimeric fusion protein with multiple binding sites for different targets. The capture agent can comprise multiple molecules each having specificity for a different target, thereby resulting in multiple capture agent-target complexes. In certain embodiments, the capture agent is comprised of proteins, such as antibodies. The capture agent can be immobilized on a solid support, such as without limitation glass, plastic, metal, latex, rubber, ceramic, polymers such as polypropylene, polyvinylidene difluoride, polyethylene, polystyrene, and polyacrylamide, dextran, cellulose, nitrocellulose, Polyvinylidene Fluoride (PVDF), nylon, amylase, and the like.

The capture agent can be directly labeled with a detectable moiety. For example, a capture agent may be directly conjugated to a detectable moiety. In the alternative, a capture agent may be detected using a secondary reagent that specifically binds to the biomarker or the capture agent-biomarker complex. Such methods are well-known in the art.

As used herein, “biomolecules” include proteins, polypeptides, nucleic acids, lipids, polysaccharides, monosaccharides, and all fragments, analogs, homologs, conjugates, and derivatives thereof.

As used herein, a “control subject” is generally an individual with no clinical signs of ASCVD, e.g., no evidence of ASCVD in imaging or other diagnostic studies such as without limitation coronary angiography, computed tomography angiogram, carotid ultrasonography, ECG stress testing (with or without imaging), etc.

In some embodiments, a biological sample from a subject may be any biological fluid, including, but not limited to, whole blood, plasma, serum, tears, saliva, mucous, cerebrospinal fluid, or urine. In some embodiments, a biological sample from a subject is any tissue sample obtained by biopsy or surgically, such as an atherosclerotic lesion.

In some embodiments, there are provided methods for diagnosing and monitoring atherosclerosis and related diseases in a subject, e.g., by imaging DSC1 expression or DSC1 protein in vivo. Many medical imaging techniques such as without limitation ultrasound, magnetic resonance imaging (MRI), X-rays, radiography, fluoroscopy, angiography, and computed tomography (CT) are known in the art, and may be used in diagnostic methods herein to detect DSC1 in a subject. Such methods may be used for example to diagnose a subject as having atherosclerosis or a related disease, or a predisposition or risk therefor; to monitor atherosclerotic disease progression; to monitor a subject's treatment or response to therapy, e.g., in a clinical trial; for epidemiological studies; and the like. In some embodiments, methods provided herein further comprise detecting an expression level of one or more additional biomarker for atherosclerotic disease in a subject.

### Kits

There are also provided herein kits comprising a DSC1 inhibitor compound or composition. Kits are generally in the form of a physical structure housing various components and may be used, for example, in practicing the methods provided herein. For example, a kit may include one or more DSC1 inhibitor disclosed herein (provided in, e.g., a sterile container), which may be in the form of a pharmaceutical composition suitable for administration to a subject. The DSC1 inhibitor can be provided in a form that is ready for use (e.g., a tablet or capsule) or in a form requiring, for example, reconstitution or dilution (e.g., a powder) prior to administration. When the DSC1 inhibitors are in a form that needs to be reconstituted or diluted by a user, the kit may also include diluents (e.g., sterile water), buffers, pharmaceutically acceptable excipients, and the like, packaged with or separately from the DSC1 inhibitors. When combination therapy is contemplated, the kit may contain several therapeutic agents separately or they may already be combined in the kit. Each component of the kit may be enclosed within an individual container, and all of the various containers may be within a single package. A kit of the present invention may be designed for conditions necessary to properly maintain the components housed therein (e.g., refrigeration or freezing). When diagnostic use is contemplated, the kit may contain reagents, solvents, buffers, etc., carrying out diagnostic methods described herein.

A kit may also contain a label or packaging insert including identifying information for the components therein and instructions for their use (e.g., dosing parameters, clinical pharmacology of the active ingredient(s), including mechanism of action, pharmacokinetics and pharmacodynamics, adverse effects, contraindications, etc., or instructions for carrying out diagnostic methods described herein). Labels or inserts can include manufacturer information such as lot numbers and expiration dates. The label or packaging insert may be, e.g., integrated into the physical structure housing the components, contained separately within the physical structure, or affixed to a component of the kit (e.g., an ampule, tube or vial). In some embodiments, kits contain instructions for use of the DSC1 inhibitor compound or composition for preventing or treating an atherosclerosis-related disorder, for preventing or treating a high-density lipoprotein (HDL) biogenesis-linked disease, disorder or condition, for promoting HDL biogenesis, or for inhibiting DSC1 in a subject.

There are further provided kits for diagnosing atherosclerosis and related disorders in a subject comprising one or more capture reagent for DSC1 and instructions for using the kit to diagnose a patient as having atherosclerosis or a related disorder when the expression level of DSC1 in a biological sample from the patient is higher than the expression level of DSC1 in a control subject. Kits may further comprise a detection reagent for detecting the capture reagent. In some embodiments, kits further comprise one or more additional capture reagent for detecting one or more additional biomarker associated with atherosclerotic disease. Kits can further comprise appropriate positive and negative controls against which a biological sample from a patient can be compared. Kits can further comprise ranges of reference values established for the expression of DSC1 in patients having atherosclerosis or related disorders or a predisposition or risk therefor.

### EXAMPLES

The present invention will be more readily understood by referring to the following examples, which are provided to illustrate the invention and are not to be construed as limiting the scope thereof in any manner.

Unless defined otherwise or the context clearly dictates otherwise, all technical and scientific terms used herein have the same meaning as commonly understood by one of ordinary skill in the art to which this invention belongs. It should be understood that any methods and materials similar or equivalent to those described herein can be used in the practice or testing of the present technology.

### Example 1. Isolation and Characterization of apoA-I-Binding Plasma Membrane (PM) Microdomains

The results described in Example 1 herein are also described in Choi, H. Y. et al., Eur Heart J 39 (14), 1194-1202 (2018), which is incorporated by reference herein in its entirety.

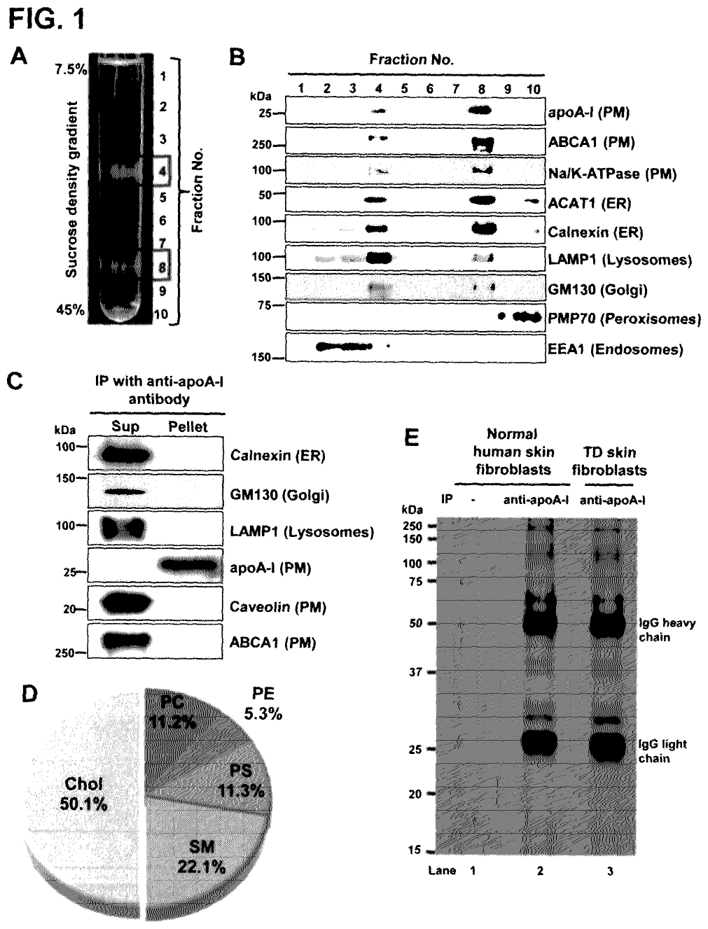

In order to isolate and characterize apoA-I-binding PM microdomains, we established a new and unbiased method using primary human skin fibroblasts (HSFs). HSF cells were incubated for 24 hours (h) with 22(R)-hydroxycholesterol/9-cis-retinoic acid to upregulate ABCA1 and promote apoA-I binding, followed by incubation with apoA-I for 1 h at 4° C. to allow apoA-I binding only to specific and initial target sites. A membrane-impermeable crosslinker, 3,3′-dithiobis(sulfosuccinimidylpropionate), was used to crosslink protein-protein interactions. The cells were homogenized and centrifuged at 3000×g to eliminate heavy subcellular organelles such as nuclei and mitochondria. The supernatant was subjected to discontinuous sucrose gradient centrifugation to separate the other subcellular organelles (Radhakrishnan, A. et al., Cell Metab. 2008; 8: 512-21). Two bands were visible after the centrifugation and 10 fractions were collected to obtain an apoA-I-enriched PM fraction (FIG. 1(A)).

PM marker proteins and apoA-I were predominantly localized in the 8^{th} fraction that also contains a large amount of endoplasmic reticulum membrane and small amounts of Golgi and lysosomal membrane proteins (FIG. 1(B)). For further purification, aggregates in the 8th fraction were dissociated by sonication prior to performing anti-apoA-I immunoprecipitation. As shown in FIG. 1(C), apoA-I was detected exclusively in the pellet devoid of endoplasmic reticulum, Golgi and lysosomal membrane markers. Furthermore, two PM proteins, caveolin and ABCA1 were excluded from the pellet, indicating that the apoA-I-associated PM domains purified under these experimental conditions were different from previously identified PM domains (ABCA1-created PM domains and caveolin-containing PM domains (caveolae)). It has been proposed previously that the ABCA1-created and caveolin-containing PM domains are required for HDL biogenesis, as they have been shown to contribute to nascent HDL formation and HDL maturation, respectively (Chao. W. T. et al., J Lipid Res. 2003; 44: 1094-9; Gu, H. M. et al., Biochim Biophys Acta. 2014; 1841: 847-58).

Throughout the purification procedure, no detergent was added, making it possible to investigate lipid composition of the purified apoA-I-associated PM domains. Total lipids extracted from the domains were analyzed for seven major lipid classes in eukaryotic plasma membranes: phosphatidylcholine (PC), phosphati dylethanolamine (PE), phosphatidylserine (PS), phosphatidylinositol (PI), phosphatidylglycerol (PG), sphingomyelin (SM), and cholesterol. FIG. 1(D) shows that the most abundant lipid was cholesterol (6.01 nM/mg cell protein+1.36) followed by SM (2.65±0.32), PS (1.35±0.38), PC (1.34±0.34), and PE (0.64±0.32). With the amount of purified sample from 1 mg cellular protein measured prior to the sucrose gradient centrifugation, PI and PG were not detectable. The enrichment of cholesterol and SM reinforces the domains as a candidate for an apoA-I binding site as cholesterol is the major target of apoA-I and HDL particles contain a high proportion of cholesterol and SM (Sorci-Thomas, M. G. et al., J Lipid Res. 2012; 53: 1890-909).

In order to determine the protein profile of the purified domains, immunoprecipitation of the 8th fraction was performed with or without anti-apoA-I antibody prior to separating the precipitated proteins on an SDS-polyacrylamide gel. Silver staining of the gel showed protein bands precipitated along with apoA-I (FIG. 1(E)). To see whether ABCA1 activity is required for apoA-I to co-precipitate the proteins, Tangier disease (TD) skin fibroblasts expressing a dysfunctional ABCA1 were compared to normal human skin fibroblasts. TD fibroblasts had only 18% of the apoA-I-mediated cholesterol efflux ability of normal fibroblasts, but the profile of co-precipitated proteins from TD fibroblasts was similar to that from normal fibroblasts (FIG. 1(E)). This finding supported the concept that the interaction between apoA-I and co-precipitated proteins is independent of ABCA1.

To identify co-precipitated proteins, the entire 2nd lane of the gel in FIG. 1(E) was processed for proteomic analysis. The proteome contained 96 proteins including the major desmosomal proteins: desmoglein (DSG) 1 and 3, desmocollin 1 (DSC1), plakophilin 1, plakoglobin, and desmoplakin (see Table 1 for the list of proteins identified by proteomic analysis of the apoA-I-associated PM microdomains purified in FIG. 1(C)). Desmosomes are located in cholesterol- and SM-rich PM domains and the assembly of desmosomes is dependent on cholesterol content (Cheng, X. et al., Mol Cell Biol. 2004; 24: 154-63; Stahley, S. N. et al., PLoS One. 2014; 9: e87809). These results suggested that apoA-I binds to a desmosomal protein.

TABLE 1
Proteins identified by proteomic analysis of the apoA-I-associated
plasma membrane microdomains purified in FIG. 1(C).

| | | Accession | Gene | MW | Peptide |
|---|---|---|---|---|---|
| Category | Protein name | number | symbol | (KDa) | count |
| Bait protein | Apolipoprotein A-I | P02647 | APOA1 | 31 | 3 |
| Desmosomal | Desmoplakin | P15924 | DSP | 332 | 101 |
| protein | Desmoglein-1 | Q02413 | DSG1 | 114 | 18 |
| | Desmoglein-3 | P32926 | DSG3 | 108 | 3 |
| | Desmocollin-1 | Q08554 | DSC1 | 100 | 9 |
| | Plakophilin-1 | Q13835 | PKP1 | 83 | 7 |
| | Junction plakoglobin | P14923 | JUP | 82 | 27 |
| | Periplakin | O60437 | PPL | 205 | 23 |
| | Envoplakin | Q92817 | EVPL | 232 | 10 |
| Lipid | Annexin A1 | P04083 | ANXA1 | 39 | 17 |
| metabolism | Annexin A2 | P07355 | ANXA2 | 39 | 18 |
| | Fatty acid-binding protein, | Q01469 | FABP5 | 15 | 6 |
| | epidermal | | | | |
| | Arachidonate 12- | O75342 | ALOX12B | 80 | 5 |
| | lipoxygenase | | | | |
| | Ganglioside GM2 activator | P17900 | GM2A | 21 | 3 |
| | Peroxiredoxin-6 | P30041 | PRDX6 | 25 | 3 |
| Calcium- and | Protein S100-A7 | P31151 | S100A7 | 11 | 5 |
| zinc-binding | Protein S100-A8 | P05109 | S100A8 | 11 | 11 |
| S100 protein | Protein S100-A9 | P06702 | S100A9 | 13 | 11 |
| | Protein S100-A11 | P31949 | S100A11 | 12 | 7 |
| | Protein S100-A16 | Q96FQ6 | S100A16 | 12 | 3 |
| | Protein S100-A14 | Q9HCY8 | S100A14 | 12 | 5 |
| Protease | Serpin B12 | Q96P63 | SERPINB12 | 46 | 11 |
| inhibitor | Serpin B3 | P29508 | SERPINB3 | 45 | 20 |
| | Serpin B5 | P36952 | SERPINB5 | 42 | 5 |
| | Cystatin-A | P01040 | CSTA | 11 | 5 |
| | Cystatin-B | P04080 | CSTB | 11 | 5 |
| | Alpha-2-macroglobulin-like | A8K2U0 | A2ML1 | 161 | 23 |
| | protein 1 | | | | |
| Protease | Caspase-14 | P31944 | CASP14 | 28 | 13 |
| | Cathepsin D | P07339 | CTSD | 45 | 13 |
| | Calpain-1 catalytic subunit | P07384 | CAPN1 | 82 | 7 |
| | Insulin-degrading enzyme | P14735 | IDE | 118 | 8 |
| | Carboxypeptidase A4 | Q9UI42 | CPA4 | 47 | 7 |
| | Bleomycin hydrolase | Q13867 | BLMH | 53 | 7 |
| | Dermcidin | P81605 | DCD | 11 | 5 |
| | Proteasome subunit alpha | O14818 | PSMA7 | 28 | 4 |
| | type-7 | | | | |
| | Proteasome subunit beta | P28072 | PSMB6 | 25 | 3 |
| | type-6 | | | | |
| | Proteasome subunit beta | P28070 | PSMB4 | 29 | 3 |
| | type-4 | | | | |
| | Proteasome subunit beta | P49720 | PSMB3 | 23 | 3 |
| | type-3 | | | | |
| | Proteasome subunit alpha | P25786 | PSMA1 | 30 | 5 |
| | type-1 | | | | |
| Antioxidant | Peroxiredoxin-1 | Q06830 | PRDX1 | 22 | 8 |
| | Peroxiredoxin-2 | P32119 | PRDX2 | 22 | 7 |
| | Catalase | P04040 | CAT | 60 | 7 |
| Ion transport | Lactotransferrin | P02788 | LTF | 78 | 7 |
| | Voltage-dependent anion- | P21796 | VDAC1 | 31 | 5 |
| | selective channel protein 1 | | | | |
| | Voltage-dependent anion- | P45880 | VDAC2 | 33 | 4 |
| | selective channel protein 2 | | | | |
| Adapter protein | 14-3-3 protein sigma | P31947 | SFN | 28 | 5 |
| | 14-3-3 protein zeta/delta | P63104 | YWHAZ | 28 | 6 |
| | 14-3-3 protein beta/alpha | P31946 | YWHAB | 28 | 3 |
| Extracellular | UPF0762 protein C6orf58 | Q6P5S2 | C6orf58 | 38 | 5 |
| vesicular | Suprabasin | Q6UWP8 | SBSN | 25 | 3 |
| exosome | Small proline-rich protein 3 | Q9UBC9 | SPRR3 | 18 | 7 |
| | BPI fold-containing family | Q96DR5 | BPIFA2 | 27 | 11 |
| | A member 2 | | | | |
| | BPI fold-containing family | Q8N4F0 | BPIFB2 | 49 | 8 |
| | B member 2 | | | | |
| | F-box only protein 50 | Q6ZVX7 | NCCRP1 | 31 | 6 |
| | Protein-glutamine gamma- | Q08188 | TGM3 | 77 | 20 |
| | glutamyltransferase E | | | | |
| | Protein-glutamine gamma- | P22735 | TGM1 | 90 | 21 |
| | glutamyltransferase K | | | | |
| | Zymogen granule protein 16 | Q96DA0 | ZG16B | 23 | 9 |
| | homolog | | | | |
| | Carbonic anhydrase 6 | P23280 | CA6 | 35 | 8 |
| | Galectin-7 | P47929 | LGALS7 | 15 | 7 |
| Others | Neuroblast differentiation- | Q09666 | AHNAK | 629 | 15 |
| | associated protein AHNAK | | | | |
| | Filaggrin | P20930 | FLG | 435 | 15 |
| | Filaggrin-2 | Q5D862 | FLG2 | 248 | 17 |
| | Hornerin | Q86YZ3 | HRNR | 282 | 15 |
| | Involucrin | P07476 | IVL | 68 | 12 |
| | Cornulin | Q9UBG3 | CRNN | 54 | 12 |
| | Ezrin | P15311 | EZR | 69 | 4 |
| | Keratinocyte proline-rich | Q5T749 | KPRP | 64 | 12 |
| | protein | | | | |
| | Polymeric immunoglobulin | P01833 | PIGR | 83 | 11 |
| | receptor | | | | |
| | Protein POF1B | Q8WVV4 | POF1B | 69 | 17 |
| | Pyruvate kinase PKM | P14618 | PKM | 58 | 12 |
| | Prolactin-inducible protein | P12273 | PIP | 17 | 7 |
| | Zinc-alpha-2-glycoprotein | P25311 | AZGP1 | 34 | 8 |
| | Polyubiquitin-B | P0CG47 | UBB | 26 | 4 |
| | Gasdermin-A | Q96QA5 | GSDMA | 49 | 7 |
| | Prelamin-A/C | P02545 | LMNA | 74 | 11 |
| | Extracellular matrix protein 1 | Q16610.2 | ECM1 | 46 | 10 |
| | Arginase-1 | P05089 | ARG1 | 35 | 7 |
| | Mucin-5B | Q9HC84 | MUC5B | 596 | 22 |
| | Mucin-7 | Q8TAX7 | MUC7 | 39 | 4 |
| | Calmodulin-like protein 5 | Q9NZT1 | CALML5 | 16 | 5 |
| | Gamma- | O75223 | GGCT | 21 | 7 |
| | glutamylcyclotransferase | | | | |
| | Histidine ammonia-lyase | P42357 | HAL | 73 | 7 |
| | Glutathione S-transferase P | P09211 | GSTP1 | 23 | 5 |
| | Thymidine phosphorylase | P19971 | TYMP | 50 | 7 |
| | Protein disulfide-isomerase | P07237 | P4HB | 57 | 6 |
| | NADP-dependent malic | P48163 | ME1 | 64 | 4 |
| | enzyme | | | | |
| | Aldehyde dehydrogenase, | P30838 | ALDH3A1 | 50 | 3 |
| | dimeric NADP-preferring | | | | |
| | Xanthine | P47989 | XDH | 146 | 3 |
| | dehydrogenase/oxidase | | | | |
| | Guanylate-binding protein 6 | Q6ZN66 | GBP6 | 72 | 6 |
| | Thioredoxin | P10599 | TXN | 12 | 3 |
| | Interleukin-1 receptor | P18510 | IL1RN | 20 | 4 |
| | antagonist protein | | | | |
| | Glutamine synthetase | P15104 | GLUL | 42 | 3 |
| | Peptidyl-prolyl cis-trans | P62937 | PPIA | 18 | 4 |
| | isomerase A | | | | |
| | Ammonium transporter Rh | Q9UBD6 | RHCG | 53 | 3 |
| | type C | | | | |
| | Adenosylhomocysteinase | P23526 | AHCY | 48 | 3 |
| | Deleted in malignant brain | Q9UGM3 | DMBT1 | 261 | 3 |
| | tumors 1 protein | | | | |

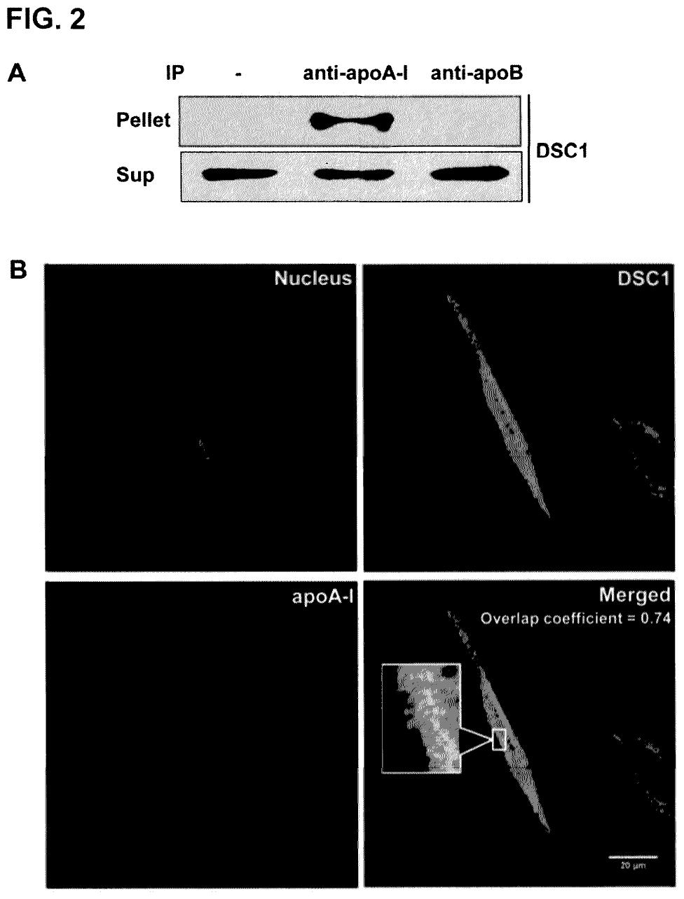

Among desmosomal proteins identified, DSC1 is dispensable for the assembly of desmosomes (Cheng, X. et al., Mol Cell Biol. 2004; 24: 154-63; Chidgey, M. et al., J Cell Biol. 2001; 155: 821-32). In addition, our results show that the cellular localization of DSC1 is not restricted to cell-cell junctions as previously reported (Myklebust, M. P. et al., Br J Cancer. 2012; 106: 756-62). These observations suggest that DSC1 may have roles other than desmosome assembly. We postulated therefore that DSC1 may be an apoA-I binding partner. To test this idea, primary HSFs were incubated with apoA-I for 1 h at 4° C. prior to lysis. The lysate was subjected to immunoprecipitation using anti-apoA-I, anti-apoB, or no antibody followed by anti-DSC1 immunoblotting. DSC1 was precipitated only by anti-apoA-I antibody (FIG. 2(A)). This binding occurred at 4° C. without a chemical crosslinker, indicating specific binding between DSC1 and apoA-I.

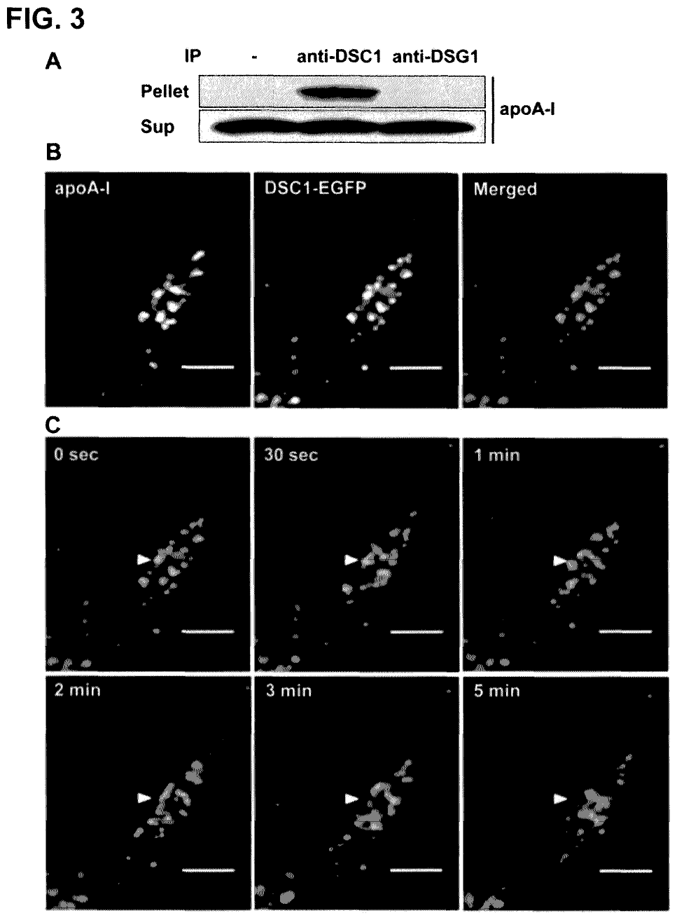

To visualize the binding, primary HSFs were incubated with Alexa Fluor 647-conjugated apoA-I for 1 h at 4° C., and fixed and stained for DSC1 using anti-DSC1 antibody. Confocal microscopic images of the cells showed a high degree of co-localization between DSC1 and apoA-I (FIG. 2(B)). For further validation, HEK293 cells transfected with DSC1b expression plasmids (pDSC1b) were incubated with apoA-I for 1 h at 37° C. prior to lysis. The lysate was subjected to immunoprecipitation using anti-DSC1, anti-DSG1, or no antibody followed by anti-apoA-I immunoblotting. This reciprocal immunoprecipitation confirmed the specificity of apoA-I-DSC1 binding (FIG. 3(A)). To see the binding in live cells, HEK293 cells expressing GFP-tagged DSC1b were incubated with Alexa Fluor 647-conjugated apoA-I for 30 min at 37° C. prior to washing unbound apoA-I out, followed by capturing time-lapse live cell images. DSC1 and apoA-I clearly co-localized in live cells (FIG. 3(B)). Time-lapse images displayed dynamic and rapid movements of the apoA-I-DSC1 complexes, for example, one of the complexes disappeared completely in 3 min (arrowheads in FIG. 3(C)). The specific binding and co-migration strongly suggest that DSC1 can regulate apoA-I function in HDL biogenesis.

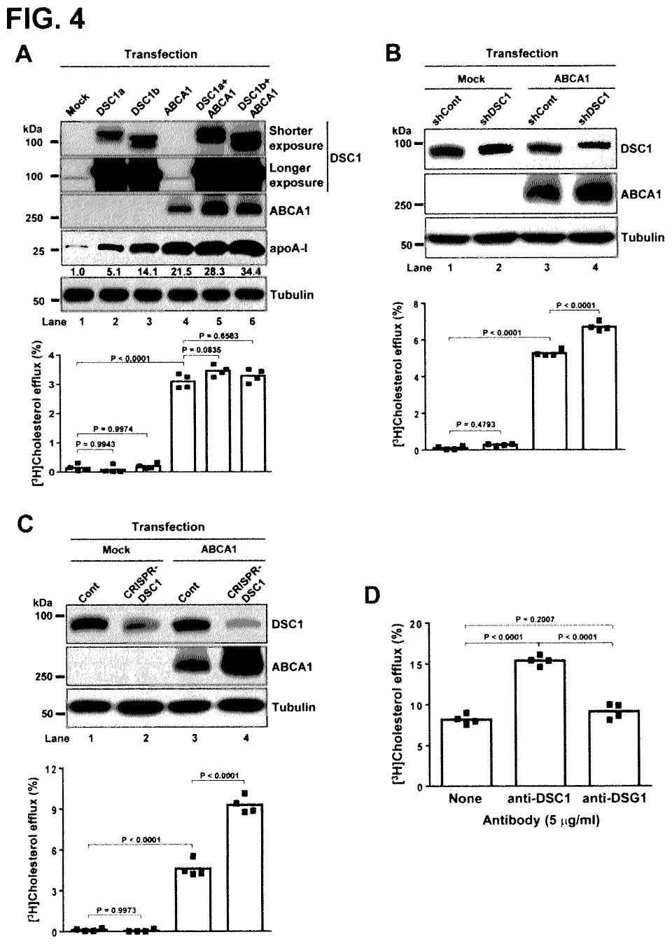

DSC1-containing PM microdomains were found to be rich in cholesterol (FIG. 1(D)). Therefore, we postulated that apoA-I-DSC1 binding may facilitate cholesterol removal by apoA-I. To test this hypothesis, an apoA-I-mediated cholesterol efflux assay was performed in HEK293 cells transfected with pDSC1. Both DSC1a and DSC1b are synthesized as preproproteins that are matured by proteolytic cleavage; a closely spaced doublet band exhibits the upper proprotein and the lower mature protein (shown in FIG. 4(A), lanes 2 and 3). HEK293 cells were found to express endogenous DSC1b (FIG. 4(A), lanes 1 and 4) and overexpression of either DSC1a or DSC1b in HEK293 significantly increased apoA-I binding capacity (FIG. 4(A), lanes 2 and 3). However, apoA-I-mediated cholesterol efflux from HEK293 cells was almost absent, regardless of the levels of DSC1 expression and DSC1-dependent apoA-I binding (FIG. 4(A), lanes 1-3). In contrast, HEK239 overexpressing ABCA1 increased both apoA-I binding and apoA-I-mediated cholesterol efflux (FIG. 4A, lane 4). Co-overexpression of ABCA1 and DSC1 showed that ABCA1- and DSC1-dependent apoA-I binding were additive, but that apoA-I-mediated cholesterol efflux from the co-overexpressing cells was not significantly different from ABCA1-only overexpressing cells (FIG. 4A, lanes 5 and 6). Of note, ABCA1 expression levels in the cells co-transfected with pABCA1-GFP/pDSC1a (FIG. 4A, lane 5) or pABCA1-GFP/pDSC1b (FIG. 4A, lane 6) were significantly higher than in the cells co-transfected with ABCA1-GFP/control plasmid (FIG. 4A, lane 4). This result is due to an inverse relationship between transfection efficiency and plasmid DNA size: transfection efficiency decreases in the order of 4.7 kb control plasmid >6.5 kb pDSC1b>6.7 kb pDSC1a>11.7 kb pABCA1-GFP, thus less pABCA1-GFP was delivered into cells in pABCA1-GFP/control plasmid versus pABCA1-GFP/pDSC1 co-transfection when the same amount of each plasmid DNA was used. Similarly, DSC1 expression levels were higher in the lanes 5 and 6 versus the lanes 2 and 3 of FIG. 4(A).

These results suggest that DSC1 and ABCA1 can independently increase apoA-I binding to cells, and that apoA-I-mediated cholesterol removal occurs through ABCA1-dependent apoA-I binding but not through DSC1-dependent apoA-I binding. Therefore, extracellular levels of apoA-I and the ABCA1/DSC1 ratio in the PM may be key determinants of HDL biogenesis.

Further, the apoA-I-DSC1 binding suggests that the DSC1 microdomain may sequester PM cholesterol, making it unavailable for efflux via the ABCA1 microdomain. DSC1 may thus function as a negative regulator of the apoA-I-mediated cholesterol removal pathway, suggesting that reducing DSC1 expression or blocking apoA-I-DSC1 interactions may enhance HDL biogenesis. We tested this hypothesis by silencing endogenous DSC1 expression in HEK293 cells. DSC1 protein levels were significantly reduced in cells stably expressing DSC1-targeting shRNAs (shDSC1) compared to cells stably expressing non-targeting control shRNAs (shCont) (FIG. 4(B), lanes 1 and 2). The near absence of apoA-I-mediated cholesterol efflux from these cells confirmed that cholesterol removal by apoA-I does not occur without ABCA1. When the cells were transfected with pABCA1-GFP, shDSC1 cells maintained higher levels of ABCA1 protein and showed greater ability to promote apoA-I-mediated cholesterol efflux, compared to shCont cells (FIG. 4(B), lanes 3 and 4).

To verify these results, the DSC1 gene in HEK293 cells was targeted using the CRISPR/Cas9 system (Malina, A. et al., Genes Dev. 2013; 27: 2602-14). This gene targeting approach achieved more effective suppression of DSC1 expression than shRNA (FIG. 4(C), lanes 1 and 2). Along with the greater reduction in DSC1, CRISPR/Cas9-mediated DSC1-targeted (CRISPR-DSC1) cells showed more effective ABCA1-dependent cholesterol efflux to apoA-I (FIG. 4(C), lanes 3 and 4) compared to shDSC1 cells (FIG. 4(B), lanes 3 and 4). Fluorescence microscopic observation of pABCA1-GFP-transfected CRISPR-DSC1 and control cells showed a similar number of GFP-positive cells, indicating that the difference in ABCA1 protein levels (FIG. 4(C), lane 3 vs. 4) was not due to the transfection efficiency of pABCA1-GFP. These DSC1 silencing studies show that the loss of DSC1 mass coincided with the gain of ABCA1 mass and function, suggesting that reduction of apoA-I-DSC1 binding increases apoA-I access to ABCA1-created apoA-I binding sites, where apoA-I protects ABCA1 from degradation and removes cholesterol for the formation of HDL particles (Wang, N. et al., J Clin Invest. 2003; 111: 99-107). If the loss of DSC1 redistributes PM cholesterol so as to increase cholesterol levels in PM microdomains containing ABCA1, ABCA1 may also be stabilized by the increased cholesterol (Hsieh, V. et al., J Biol Chem. 2014; 289: 7524-36).

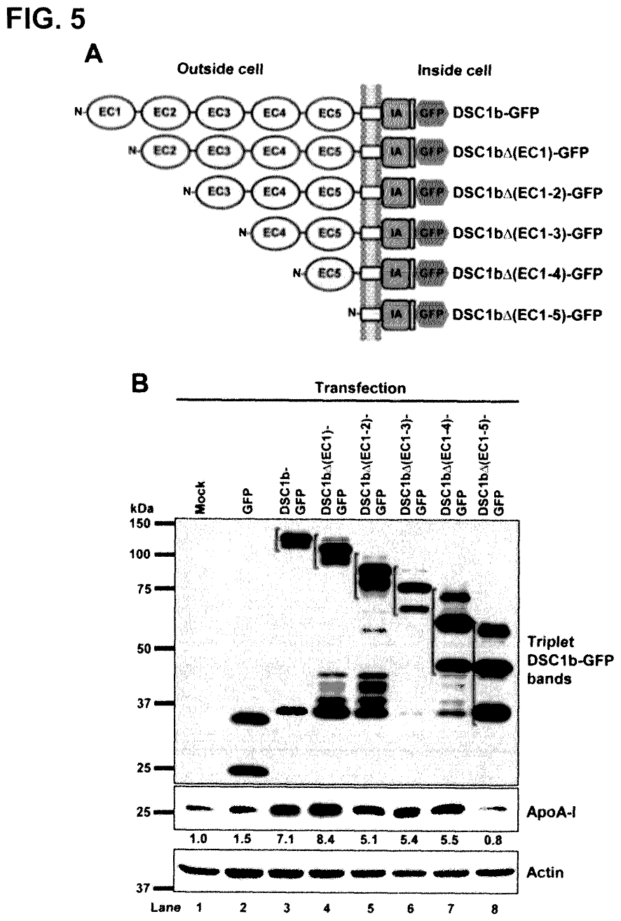

To test if inhibition of apoA-I-DSC1 interactions is sufficient to promote ABCA1-dependent cholesterol efflux to apoA-I, primary HSFs were treated with anti-DSC1 antibody for 1 h prior to performing apoA-I-mediated cholesterol efflux assay. As seen in FIG. 4(D), cells pre-treated with an anti-DSC1 antibody directed against a portion (amino acid residues 424-547 of mature DSC1) of the DSC1 extracellular region markedly enhanced cholesterol efflux to apoA-1, whereas an anti-DSG1 antibody developed against whole DSG1 protein had no effect. These results suggest that apoA-I may bind within the residues 424-547 of mature DSC1. The DSC1 extracellular region comprises 5 extracellular cadherin repeats (EC1-5) (Kowalczyk, A. P. and Green, K. J., Prog Mol Biol Transl Sci. 2013; 116: 95-118) and the residues 424-547 correspond to a part of the EC4 plus the entire EC5 repeats, suggesting that the EC4 and/or EC5 repeats may be responsible for the binding of apoA-I. Considering the large size of antibody molecules that are approximately 150 kDa and glycosylated, it is also conceivable that steric hindrance induced by antibody binding to the EC4 and/or EC5 repeats may have interfered with apoA-I binding to EC1-3 repeats. To investigate which EC repeat of DSC1 binds apoA-I, plasmids encoding a series of truncated DSC1b proteins lacking EC1 to EC1-5 repeats were constructed (FIG. 5(A)). HEK293 cells overexpressing full-length or truncated DSC1b-GFP protein were incubated with apoA-I for 1 h at 37° C. prior to extensive washing and lysis of the cells. The lysate was subjected to anti-GFP immunoblotting to determine DSC1b-GFP expression levels and anti-apoA-I immunoblotting to measure the apoA-I amount bound to the cells. As DSC1 is synthesized as a preproprotein, the triplet DSC1b-GFP bands exhibit the largest preproprotein, the intermediate proprotein and the smallest mature protein (FIG. 5(B)). As shown in FIG. 5(B), a markedly increased amount of apoA-I bound to cells overexpressing the full-length DSC1b-GFP (lane 3) compared to control cells (lanes 1 and 2). The DSC1b-dependent apoA-I binding capacity was increased slightly by deleting the EC1 repeat (lane 4) but decreased moderately by deleting the EC1-2 repeats (lane 5), suggesting that the EC2 repeat plays a role in apoA-I binding. A similar apoA-I binding capacity to the EC1-2 (lane 5), EC1-3 (lane 6) or EC1-4 (lane 7) repeats-deleted DSC1bs suggests that the EC3 and 4 repeats play no role in apoA-I binding. Deletion of the EC1-5 repeats completely abolished the DSC1b-dependent apoA-I binding (lane 8), suggesting that the EC5 repeat plays an essential role in apoA-I-DSC1 interactions. The EC5 repeat, comprised of eighty amino acid residues (459-538 of mature DSC1), is therefore a novel therapeutic target for the promotion of HDL biogenesis.

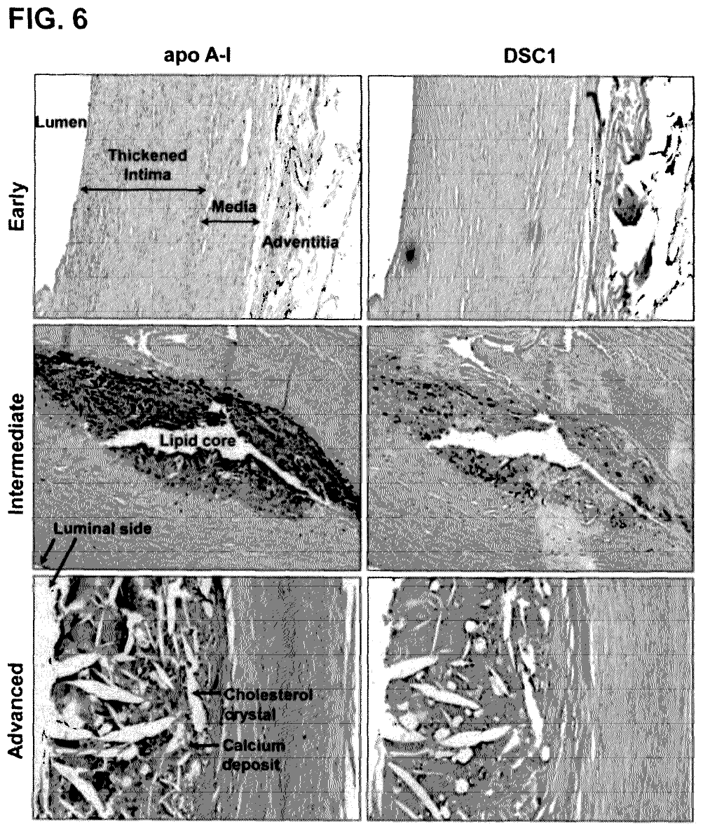

The relevance of apoA-I-DSC1 binding to human atherosclerosis was investigated by performing immunohistochemical staining for apoA-I and DSC1 on coronary artery sections obtained from patients with coronary atherosclerosis. Early-stage atherosclerotic lesions characterized by intimal thickening were weakly and sparsely stained for apoA-I and DSC1 (FIG. 6). In intermediate-stage lesions, densely concentrated apoA-I staining in the lipid core periphery overlapped with DSC1 staining (FIG. 6), suggesting that apoA-I-DSC1 binding indeed occurs in coronary atherosclerotic lesions in humans. Advanced-stage lesions characterized by the presence of cholesterol crystals and calcium exhibited a large amount of both apoA-I and DSC1 in a necrotic lipid core (FIG. 6). The cellular architecture observed in apoA-I/DSC1-positive intermediate lesions was no longer present in apoA-I/DSC1-positive advanced lesions (FIG. 6), suggesting that arterial cells co-localizing apoA-I and DSC1 likely die and contribute to the formation of cholesterol-laden necrotic cores.

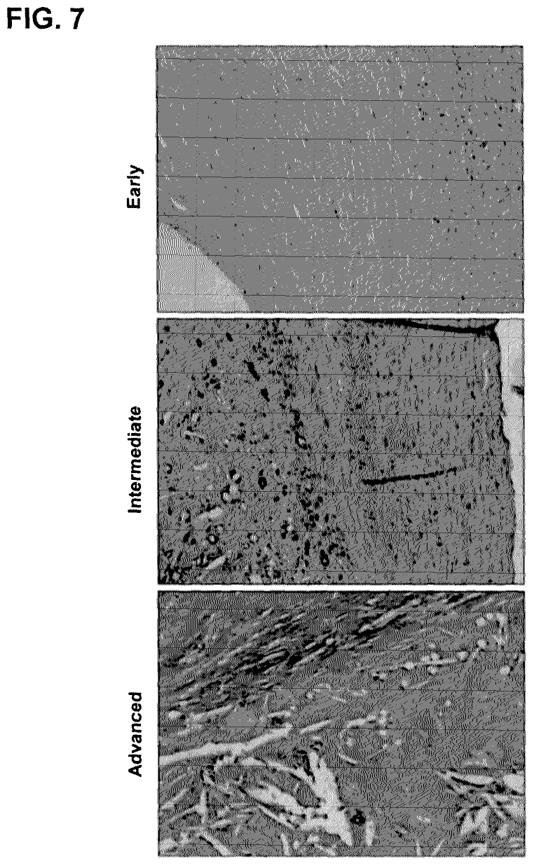

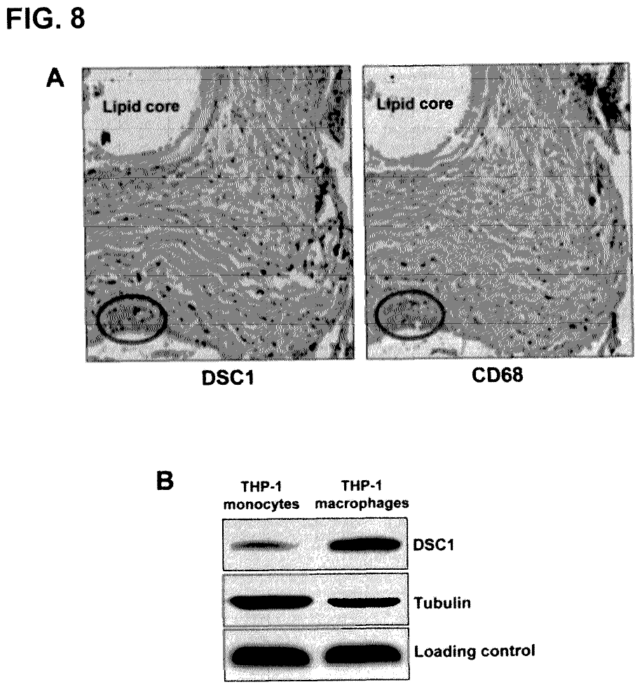

The association between increased DSC1 expression levels and lesion progression was also observed in human carotid atherosclerosis (FIG. 7), suggesting that DSC1 may play important roles in the development of atherosclerotic lesions in general. In support, DSC1 was expressed in CD68-immunopositive cells that play crucial roles in all stages and sites of atherosclerosis (FIG. 8(A)) (Stoger, J. L. et al., Atherosclerosis. 2012; 225: 461-8). CD68 is a pan-macrophage marker and anti-DSC1 immunoblotting showed that differentiation of human THP-1 monocytes into macrophages was associated with upregulation of DSC1 expression (FIG. 8(B)). These results suggest that DSC1 expression in macrophages in atherosclerotic lesions may drive prevention of HDL biogenesis, cholesterol deposition, cell death and thus disease progression.

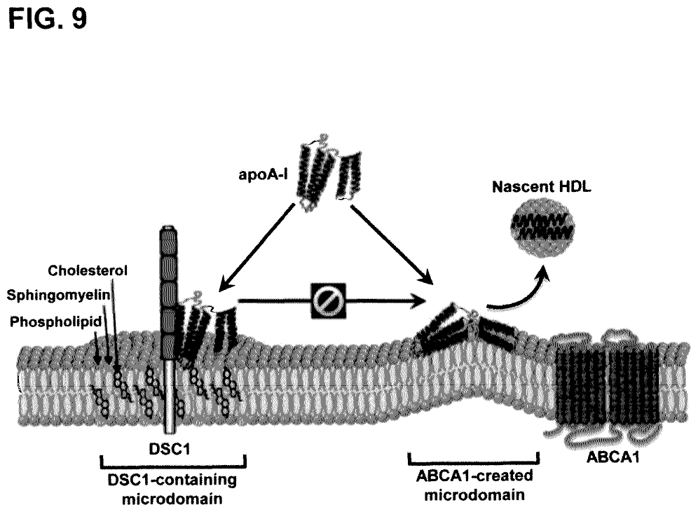

In sum, these results show that DSC1 located in cholesterol- and SM-rich microdomains binds apoA-I and prevents apoA-I from forming HDL particles. DSC1-containing microdomains therefore counteract ABCA1-created microdomains that facilitate apoA-I binding for HDL formation. These two apoA-I binding but functionally opposing PM microdomains may regulate HDL biogenesis and PM cholesterol levels. An illustration of this model is shown in FIG. 9.

In keeping with the general idea that DSC1 is dispensable for the assembly of desmosomes, and may harbor its essential functional elements in the extracellular and transmembrane domains, we have shown here that apoA-I binds to the DSC1 extracellular domain (FIG. 5), suggesting that a role of DSC1 in desmosomes may be to prevent HDL biogenesis for the conservation of PM cholesterol. It is noted that DSC1 is most abundantly expressed in the skin and Dsc1^{-/-} mice show defects in skin barrier function, suggesting that DSC1-dependent maintenance of high cholesterol levels in desmosomes may be necessary for the formation of a water-impermeable skin barrier.

It is widely believed that desmosomes are largely confined to epithelia and cardiac muscle, and are absent from leucocytes and endothelia, therefore our demonstration of DSC1 expression in macrophages (FIG. 8) and atherosclerotic plaques (FIGS. 6 and 7) is novel. DSC1 expression in arterial intima could be a maladaptive process: DSC1-containing desmosomes may be assembled to maintain intimal tissue integrity or repair damaged intima, but result in building up cholesterol by impairing HDL biogenesis. Considering that atherosclerosis is a cholesterol deposition-driven chronic inflammatory disease and that HDL biogenesis is the major mechanism to remove excess cholesterol from macrophages, our results suggest that DSC1-attributed impairment of HDL biogenesis from intimal macrophages is a highly likely contributor to the progression of atherosclerosis.

It has been reported that atherosclerotic plaque-laden human aorta contains at least 100-fold more apoA-I compared to normal aorta, and that the vast majority of apoA-I within the plaque is functionally impaired and not associated with HDL particles (DiDonato, J A et al., Circulation 2013, 128: 1644-1655). Our findings suggest that impaired HDL biogenesis owing to apoA-I-DSC1 binding could be the underlying mechanism for the massive accumulation of dysfunctional apoA-I. The sequestration of apoA-I within the atherosclerotic plaque may render the reverse cholesterol transport defective and thus contribute to low levels of circulating HDL in atherosclerotic cardiovascular disease.

Finally, our results show that DSC1 knockdown or blocking antibodies increased HDL biogenesis (FIGS. 4(B)-(D)) and that the EC2 and EC5 repeats of DSC1 mediate apoA-I-DSC1 interactions (FIG. 5). These results suggest that agents such as monoclonal antibodies or small molecules that inhibit apoA-I binding to the EC2 and/or EC5 repeats of DSC1 can be effective therapeutics for promoting HDL biogenesis and preventing or treating atherosclerosis, disorders of defective cholesterol homeostasis, and related disorders.

### Example 2. Further Characterization of the apoA-I Binding Site in DSC1

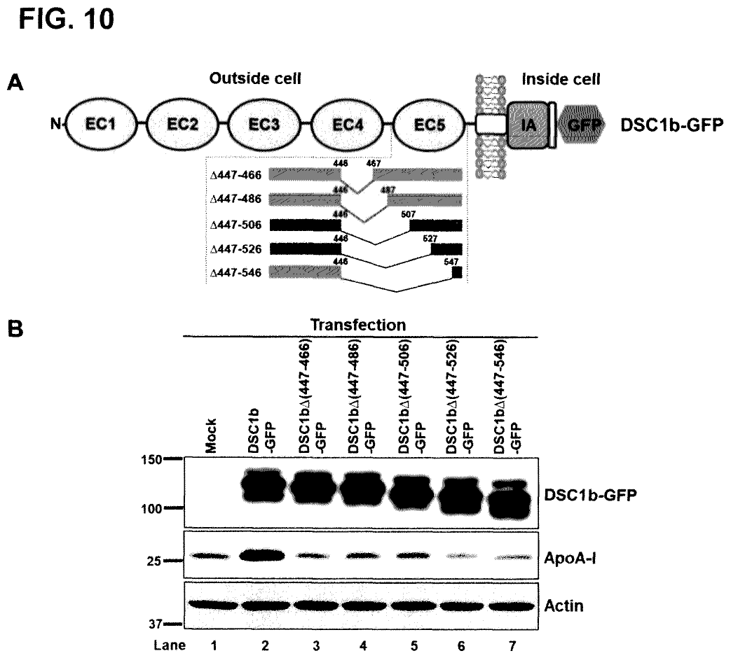

In Example 1 above, we described the identification of Desmocollin 1 (DSC1) as a novel apoA-I binding protein, and mutational analysis studies showing that, among the five extracellular cadherin repeats (EC1-5) of DSC1 protein, the EC5 repeat comprised of 80 amino acid residues (459-538) was essential for the interactions between apoA-I and DSC1. Next, to narrow down the apoA-I binding site in the EC5, plasmids encoding progressive EC5 deletion mutants were constructed (FIG. 10(A)). HEK293 cells were transfected with the constructs to express the full-length DSC1b or a series of EC5 deletion mutants. The cells were incubated with apoA-I prior to determining the effect of progressive EC5 deletions on apoA-I binding (FIG. 10(B)). Expression of the full-length DSC1b markedly increased apoA-I binding capacity (FIG. 10(B), lanes 1 and 2), but the DSC1b-dependent apoA-I binding was not observed in cells expressing DSC1bΔ447-466 (FIG. 10(B), lanes 2 and 3). The complete abolishment of DSC1 effect on apoA-I binding suggested that the 20 residues (447-466) were crucial for apoA-I-DSC1 interactions.

Amino acid numbering starts at the amino-terminal amino acid of mature DSC1 protein herein. It should be noted that immature (preproprotein) DSC1 is cleaved to remove the amino-terminal 134 amino acids, producing mature DSC1.

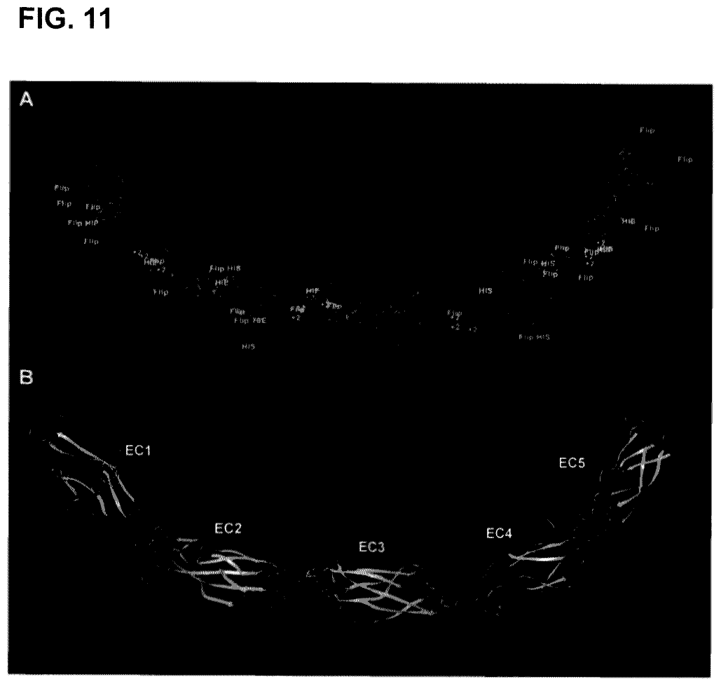

To investigate if the 20 residues are involved in creating a protein binding site, we analyzed a crystal structure of the human DSC1 ectodomain (protein data bank ID=5IRY) imported from the RCSB protein data bank (https://www.rcsb.org). Due to the limited resolution of crystallography, a protein crystal structure in its raw state is not suitable for molecular modeling. Common problems include missing atoms and incorrect bond orders, protonation states and charges, or orientations of chemical groups. To prepare the DSC1 crystal structure for use in molecular modeling, we used the Protein Preparation Wizard in the Schrodinger software graphical user interface called Maestro (version 11.0). The Wizard augmented DSC1 crystal data by fixing structural defects, removing unwanted molecules and optimizing DSC1 structure. The first step was to ensure the chemical correctness of DSC1 by correcting defective bond order assignments, adding missing hydrogens, creating zero-order bonds to metals, creating disulfide bonds, filling in missing side chains, and capping termini. In the second step of review and modification, dimeric DSC1 structure was reduced to monomer. Also, ionization or tautomeric states of co-crystalized heteroatom groups such as ions and cofactors were corrected. In the final refinement step, hydrogen-bond assignment was optimized, water molecules with less than 3 hydrogen-bonds to non-waters were removed, and the corrected structure was minimized to alleviate any significant steric clashes. The finalized DSC1 structure for molecular modeling is shown in FIG. 11.

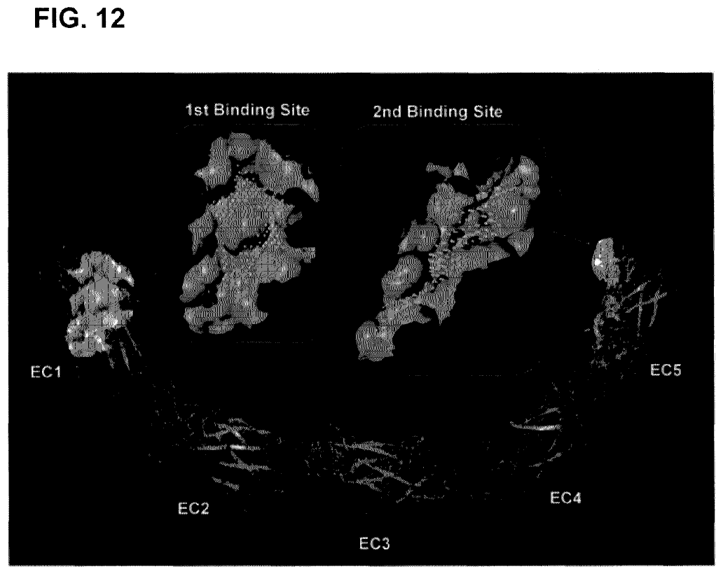

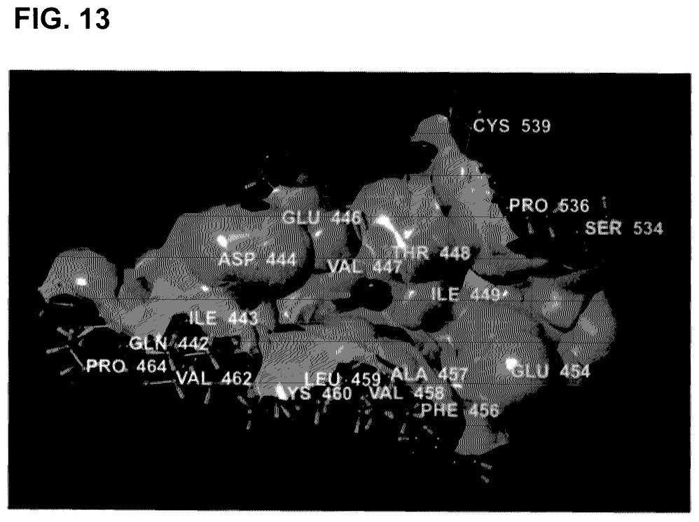

The presence of protein binding sites in the DSC1 was calculated by the SiteMap tool in Maestro. Binding sites identified by the SiteMap's algorithm were represented as collections of site points at or near the surface of DSC1 that are contiguous or separated in solvent-exposed regions by short gaps that could plausibly be spanned by ligand functionality. To visualize binding site features, a grid of points to identify potential hydrophobic and hydrophilic regions was used; the hydrophilic regions were further classified into hydrogen-bond donor and hydrogen-bond acceptor regions, and the binding site surface was contoured. Based on binding site properties such as size, functionality and extent of solvent exposure, an overall SiteScore that assesses a site's propensity for ligand binding was calculated in order to rank possible binding sites. The highest-scoring binding site was found in the EC1 and the second one in the EC5 (FIG. 12). Desmosomal cadherin proteins including DSC1 are known to bind through their EC1 repeats in order to form hemophilic or heterophilic dimers (Nie, Z. et al., J Biol Chem 286, 2143-2154 (2011); Harrison, O. J., et al., Proc Natl Acad Sci USA 113, 7160-7165 (2016)), indicating that binding sites identified by the SiteMap may be reliable. There is no known protein interacting with the second binding site located in the EC5, but interestingly amino acid residues comprising the binding site within a radius of 3 Å are largely coincided with the 20 residues (447-466) that are crucial for apoA-I-DSC1 interactions (FIG. 13). These results strongly suggest that apoA-I may bind to the site and that chemical compounds being able to bind to the site may block apoA-I-DSC1 interactions.

### Example 3. Identification of Chemical Compounds Inhibiting apoA-I-DSC1 Binding

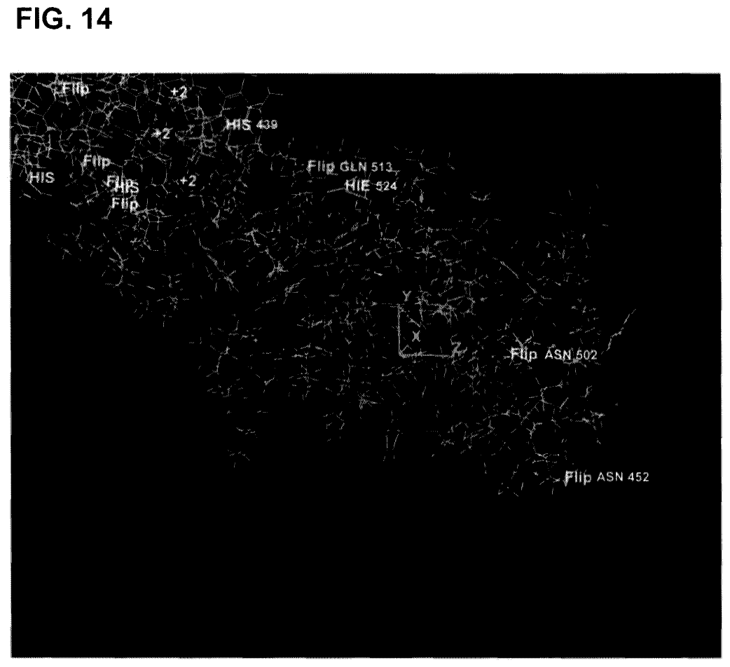

In order to identify chemical compounds that inhibit apoA-I-DSC1 binding, the physical properties of the volume of the predicted apoA-I binding site were specified using the Receptor Grid Generation panel in Maestro. The van der Waals radii of nonpolar DSC1 atoms were left unchanged by setting the scaling factor of van der Waals radius at 1.0; nonpolar was defined by the partial atomic charge less than 0.25. A grid area encompassing the binding site was calculated and enclosed by a box at the centroid of SiteMap points (FIG. 14). The grid represents the active site of DSC1 for chemical compound (ligand) docking jobs.

Databases of commercially-available chemical compounds are freely downloadable, and we obtained structure data (SD) for approximately 10 million compounds in the SD file format from the Selleckchem (http://www.selleckchem.com), Enamine (http://www.enamine.net) and ZINC (http://zinc.docking.org) compound libraries. To screen the compounds in search of potential ligands for the DSC1 grid, ligand docking analysis was carried out using Glide (grid-based ligand docking with energetics) in Maestro. To achieve the best results of Glide docking analysis, each ligand structure must be three-dimensional, have realistic bond lengths and bond angles, consist of a single molecule that has no covalent bond to the receptor, have all its hydrogens, and have an appropriate protonation state for physiological pH values. The preparation of ligand structures for Glide was done using the LigPrep panel in Maestro. In cases of complex ligands, LigPrep produced multiple output structures for a single input structure by generating different protonation states, stereochemistry, tautomers, and ring conformations.

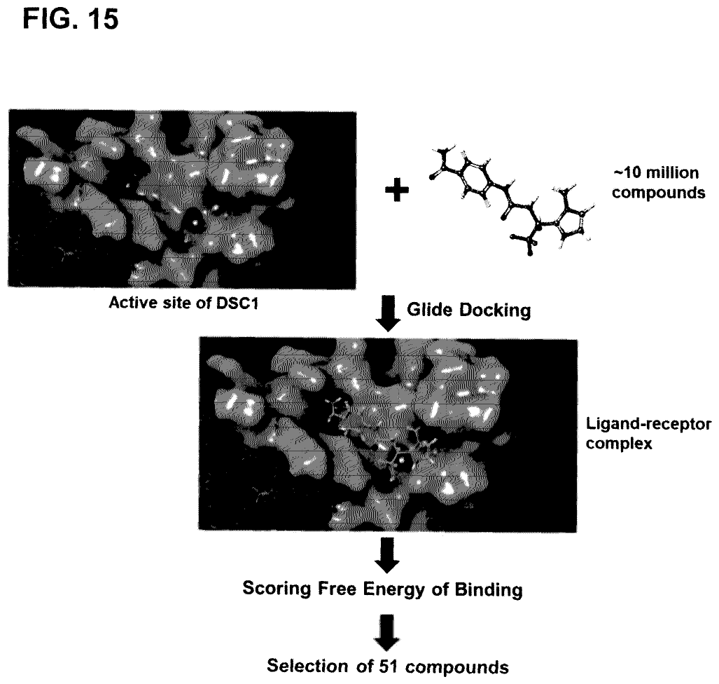

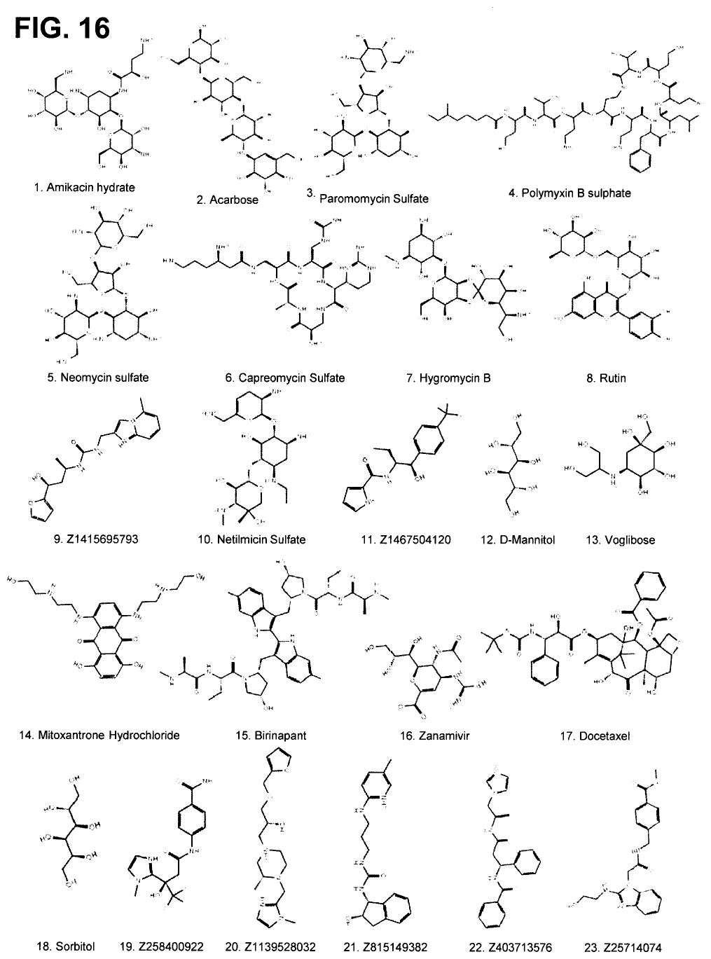

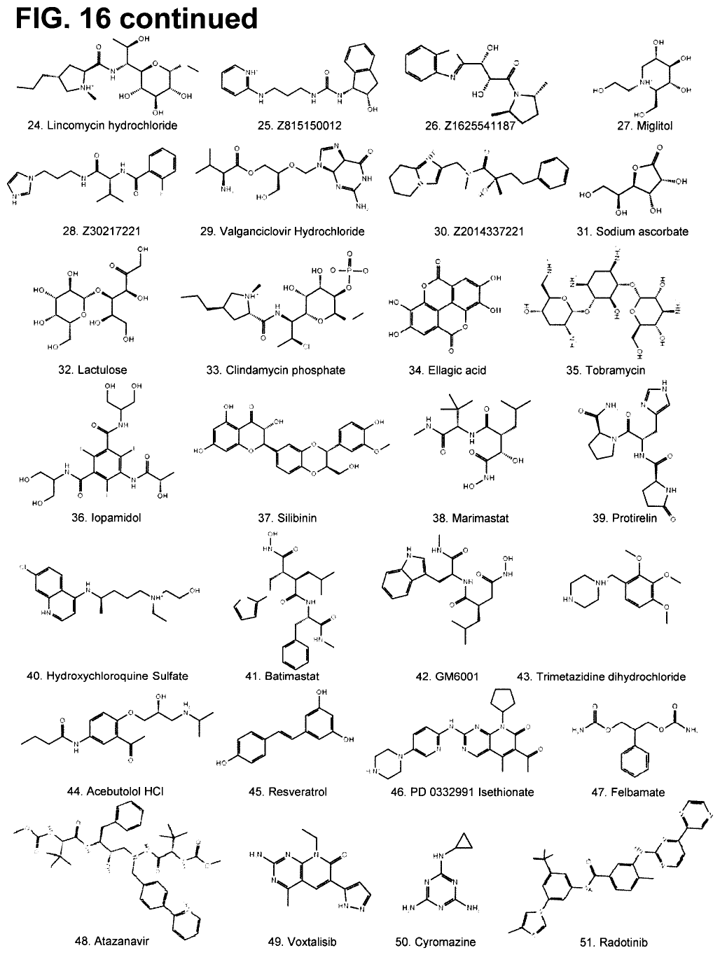

To calculate computational docking of the ligands prepared by the LigPrep into the DSC1 grid, the Glide Ligand Docking panel in Maestro was used. Glide performs a systemic search of the conformational, orientational and positional space of the docked ligand in order to generate an accurate pose for each ligand-receptor complex. Ligand-receptor interactions such as hydrogen bonds and hydrophobic contacts are scored to estimate the free energy of ligand binding. Based on the binding free energies, ligands that favorably interact with the receptor are rank-ordered. To decrease penalties for close ligand-receptor contacts, the van der Waals radii of nonpolar ligand atoms were scaled by 0.8; nonpolar was defined by the partial atomic charge less than 0.15. The docking job was performed with the setting of docking ligands flexibly, penalizing amide C—N bonds that are not cis or trans conformation, and adding Epik ionization and tautomeric state penalties to docking score. After performing virtual screening of ligands with the standard-precision docking method, the top-ranked 10% of ligand poses were reanalyzed by the extra-precision docking method. We used extra-precision docking score and docking pose to choose 51 favorable ligands for the active site of DSC1. The overall screening work-flow is shown schematically in FIG. 15. Chemical structures of the selected 51 compounds are shown in FIG. 16. Chemical information and the extra-precision docking scores of the 51 compounds are shown in Table 2.

TABLE 2
Chemical formulae and docking scores of 51 compounds selected as potential ligands
for the active site of DSC1. The three most active compounds in biological
assays for promotion of apoA-I-mediated HDL biogenesis are shown in bold.

| | | | Mol. | | Docking |
|---|---|---|---|---|---|
| No. | Compound Name | Formula | Weight | CAS Number | Score |
| 1 | Amikacin hydrate | C22H45N5O14R | 603.6 | 1257517-67-1 | −11.763 |
| 2 | Acarbose | C25H43NO18 | 645.6 | 56180-94-0 | −10.291 |
| 3 | Paromomycin | C23H47N5O18S | 713.7 | 1263-89-4 | −9.179 |
| | Sulfate | | | | |
| 4 | Polymyxin B | C56H100N16O17S | 1301.6 | 1405-20-5, | −8.949 |
| | sulphate | | | 4135-11-9 | |
| 5 | Neomycin sulfate | C23H48N6O17S | 712.7 | 1405-10-3 | −8.82 |
| 6 | Capreomycin | C25H44N14O7R | 652.7 | 1405-37-4 | −8.37 |
| | Sulfate | | | | |
| 7 | Hygromycin B | C20H37N3O13 | 527.5 | 31282-04-9 | −7.937 |
| 8 | Rutin | C27H30O16 | 610.5 | 153-18-4 | −7.885 |
| 9 | Z1415695793 | C18H22N4O3 | 342.4 | Not registered | −7.764 |
| 10 | Netilmicin Sulfate | C42H92N10O34S5 | 1441.6 | 7664-93-9 | −7.584 |
| 11 | Z1467504120 | C16H17F3N2O2 | 326.3 | Not registered | −7.407 |
| 12 | D-Mannitol | C6H14O6 | 182.2 | 69-65-8 | −7.379 |
| 13 | Voglibose | C10H21NO7 | 267.3 | 83480-29-9 | −7.335 |
| 14 | Mitoxantrone | C22H30C12N4O6 | 517.4 | 70476-82-3 | −7.325 |
| | Hydrochloride | | | | |
| 15 | Birinapant | C42H56F2N8O6 | 806.9 | 1260251-31-7 | −7.239 |
| 16 | Zanamivir | C12H20N4O7 | 332.3 | 139110-80-8 | −7.147 |
| 17 | Docetaxel | C43H53NO14 | 807.9 | 114977-28-5 | −7.076 |
| 18 | Sorbitol | C6H14O6 | 182.2 | 50-70-4 | −6.833 |
| 19 | Z258400922 | C15H15F3N4O3 | 356.3 | Not registered | −6.795 |
| 20 | Z1139528032 | C18H28N4O3 | 348.4 | Not registered | −6.718 |
| 21 | Z815149382 | C19H24N4O2 | 340.4 | Not registered | −6.667 |
| 22 | Z403713576 | C22H24N4O2 | 376.5 | Not registered | −6.653 |
| 23 | Z25714074 | C20H22N4O4 | 382.4 | Not registered | −6.641 |
| 24 | Lincomycin | C18H35ClN2O6S | 443.0 | 859-18-7 | −6.632 |
| | hydrochloride | | | | |
| 25 | Z815150012 | C18H22N4O2 | 326.4 | Not registered | −6.608 |
| 26 | Z1625541187 | C16H20N2O3S | 320.4 | Not registered | −6.579 |
| 27 | Miglitol | C8H17NO5 | 207.2 | 72432-03-2 | −6.56 |
| 28 | Z30217221 | C18H23FN4O2 | 346.4 | Not registered | −6.545 |
| 29 | Valganciclovir | C14H25ClN6O5 | 392.8 | 175865-59-5 | −6.485 |
| | Hydrochloride | | | | |
| 30 | Z2014337221 | C20H27N3O2 | 341.5 | Not registered | −6.416 |
| 31 | Sodium ascorbate | C6H10NaO6 | 201.1 | 134-03-2 | −6.287 |
| 32 | Lactulose | C12H22O11 | 342.3 | 4618-18-2 | −6.237 |
| 33 | Clindamycin | C18H34ClN2O8PS | 505.0 | 24729-96-2 | −6.215 |
| | phosphate | | | | |
| 34 | Ellagic acid | C14H6O8 | 302.2 | 476-66-4 | −6.068 |
| 35 | Tobramycin | C18H37N5O9 | 467.5 | 32986-56-4 | −5.883 |
| 36 | Iopamidol | C17H22I3N3O8 | 777.1 | 60166-93-0 | −5.839 |
| 37 | Silibinin | C25H22O10 | 482.4 | 22888-70-6 | −5.811 |
| 38 | Marimastat | C15H29N3O5 | 331.4 | 154039-60-8 | −5.734 |
| 39 | Protirelin | C16H22N6O4 | 362.4 | 24305-27-9 | −5.157 |
| 40 | Hydroxychloroquine | C18H28ClN3O5S | 434.0 | 747-36-4 | −4.585 |
| | Sulfate | | | | |
| 41 | Batimastat | C23H31N3O4S2 | 477.6 | 130370-60-4 | −4.387 |
| 42 | GM6001 | C20H28N4O4 | 388.5 | 142880-36-2 | −4.302 |
| 43 | Trimetazidine | C14H24Cl2N2O3 | 339.3 | 13171-25-0 | −4.261 |
| | dihydrochloride | | | | |
| 44 | Acebutolol HCl | C18H29ClN2O4 | 372.9 | 34381-68-5 | −3.98 |
| 45 | Resveratrol | C14H12O3 | 228.2 | 501-36-0 | −3.974 |
| 46 | PD 0332991 | C26H35N7O6S | 573.7 | 827022-33-3, | −3.417 |
| | Isethionate | | | 571190-30-2 | |
| 47 | Felbamate | C11H14N2O4 | 238.2 | 25451-15-4 | −3.35 |
| 48 | Atazanavir | C38H52N6O7 | 704.9 | 198904-31-3 | −3.292 |
| 49 | Voxtalisib | C13H14N6O | 270.3 | 934493-76-2 | −3.274 |
| 50 | Cyromazine | C6H10N6 | 166.2 | 66215-27-8 | −3.121 |
| 51 | Radotinib | C27H21F3N8O | 530.5 | 926037-48-1 | −3.042 |

### Example 4. DSC1 Inhibitor Compounds Promote HDL Biogenesis

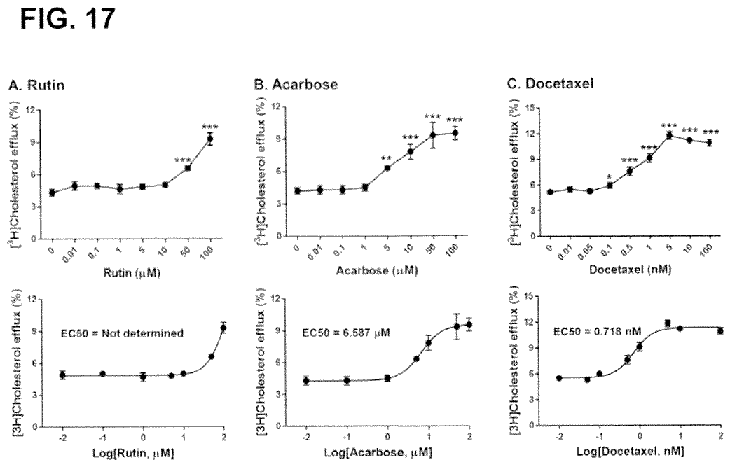

To investigate the biological activity of the 51 compounds identified in Example 3 in modulating HDL biogenesis, we performed an apoA-I-mediated cholesterol efflux study as described previously (see Choi, H. Y. et al., J Biol Chem 278, 32569-32577 (2003)). We found that 3 compounds were particularly active in promoting HDL biogenesis (Table 2). Dose-response curves for the 3 most active compounds showed that the most potent compound was docetaxel having the half-maximal effective concentration (EC50) of 0.72 nM (FIG. 17). The second most potent compound was Acarbose, and the third most potent compound was Rutin.

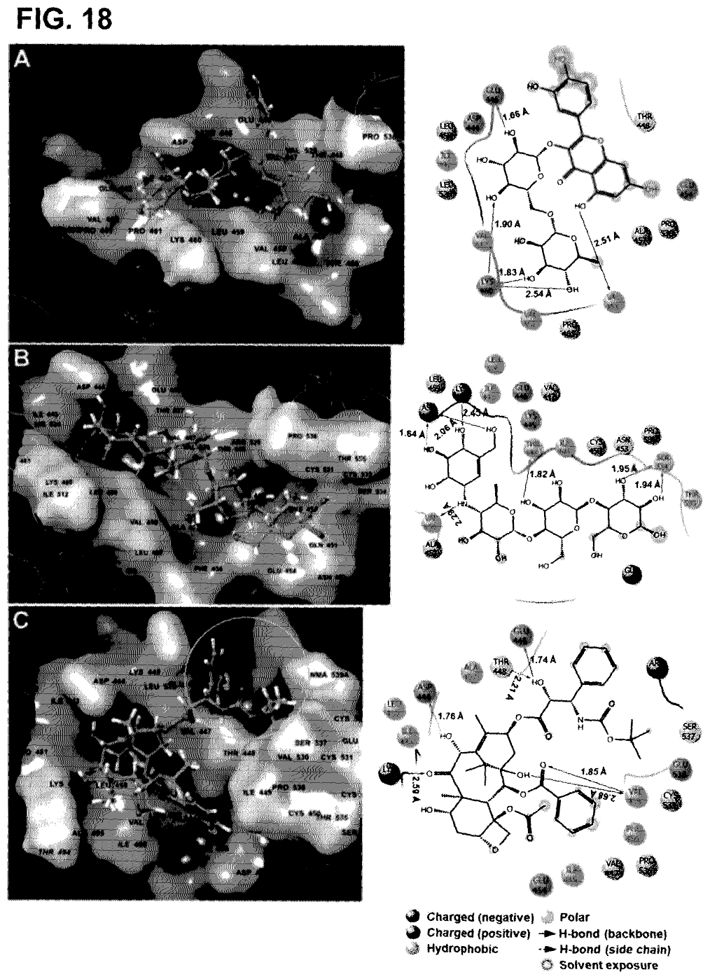

The active site of DSC1 is featured by an abundance of hydrogen-bond acceptor regions displayed in blue in FIG. 13, and all three of the most active compounds (rutin, acarbose and docetaxel) are enriched with hydrogen-bond donor groups shown in red in FIG. 16. These results suggest that hydrogen bonds are the most important interactions between the DSC1 active site and an active compound. Among the three most active compounds, rutin was the least potent in promoting HDL biogenesis (FIG. 17(A)) and its Glide docking score was −7.89 (Table 2). Based on the DSC1 crystal structure 5IRY, rutin was simulated to form three strong hydrogen bonds with Glu446 (the distance of hydrogen bond: 1.66 Å) and Lys460 (1.90 Å and 1.83 Å), and two moderate hydrogen bonds with Lys460 (2.54 Å) and Val458 (2.51 Å), as displayed in FIG. 18(A). Acarbose, having an EC50 value of 6.59 μM (FIG. 17(B)) and a Glide docking sore of −10.29 (Table 2), was simulated to form six strong hydrogen bonds with Asp444 (1.64 Å and 2.06 Å), Thr448 (1.82 Å), Val458 (2.29 Å) and Ser534 (1.94 Å and 1.95 Å), and one moderate hydrogen bond with Lys460 (2.45 Å), as displayed in FIG. 18(B). Docetaxel, showing the highest potency in this study, had an EC50 of 0.72 nM (FIG. 17C) and a Glide docking score of −7.08 (Table 2). Docetaxel was simulated to form four strong hydrogen bonds with Asp444 (1.76 Å), Glu446 (1.74 Å), Thr448 (2.21 Å) and Val458 (1.85 Å), and two moderate hydrogen bonds with Lys460 (2.59 Å) and Val458 (2.68 Å), as displayed in FIG. 18(C). All of the three most active compounds had hydrogen bond interactions with Val458 and Lys460, and two of the three compounds with Asp444, Glu446 and Thr448. These five DSC1 residues may therefore play central roles in interacting with apoA-I. Rutin was predicted to form hydrogen bonds with three residues in the DSC1 active site, while acarbose and docetaxel were predicted to form hydrogen bonds with five residues in the DSC1 active site (FIG. 18), suggesting that the potency of a compound may depend on the number and the location of residues with which the compound is able to form hydrogen bonds.

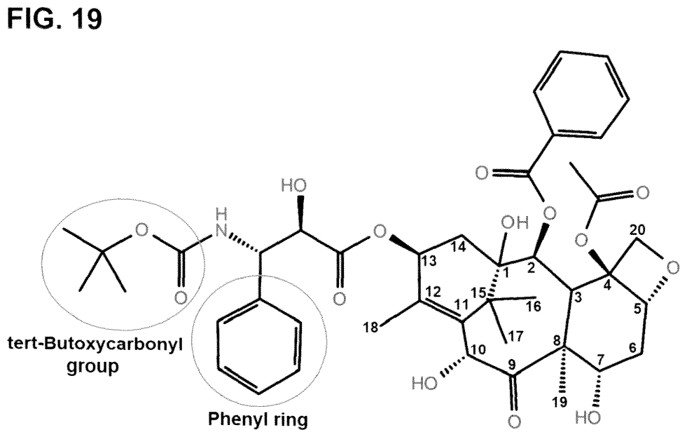

One of the chief differences between acarbose and docetaxel is that docetaxel is predicted to interact with additional binding cavities indicated by a yellow circle in FIG. 18(C). Docetaxel is composed of a taxane ring with an ester sidechain attached at carbon (C)-13 of the taxane ring (FIG. 19; Mastropaolo, D. et al., Proc Natl Acad Sci USA 92, 6920-6924 (1995)). The C-13 sidechain contains the phenyl ring and the tert-butoxycarbonyl group. The two chemical groups are simulated to interact with binding cavities that were not included in the apoA-I binding site seen in FIG. 13. A hydroxyl group positioned immediately before the phenyl ring forms two strong hydrogen bonds with Glu446 (1.74 Å) and Thr448 (2.21 Å) as displayed in FIG. 18(C), which may lead or stabilize the interactions between the C-13 sidechain and the cavities. Among nine hydrophobic residues displayed in each of the three ligand interaction diagrams, six residues (Ile443, Val447, Ala457, Val458, Leu459 and Pro536) were common for all three of the most active compounds (FIG. 18), suggesting that hydrophobic interactions may also contribute to enhancing compound activities.

In summary, docetaxel was identified as a potent promoter of apoA-I-mediated HDL biogenesis with an EC50 value of 0.72 nM in a cell-based assay (FIG. 17). Mutational analysis of the apoA-I binding site in the EC5 region of DSC1 (FIG. 10) and computational mapping of protein binding sites in DSC1 (FIGS. 12-13) suggested that there is an apoA-I binding site in the EC5 and that docetaxel can promote HDL biogenesis by binding to the apoA-I binding site and thus inhibiting apoA-I-DSC1 interactions. The taxane ring of docetaxel is predicted to dock to the apoA-I binding site through hydrogen bond and hydrophobic interactions. Acarbose and rutin are also predicted to dock to the same binding site (FIG. 18), suggesting that the high potency of docetaxel was not solely dependent on the apoA-I binding site. Our Glide docking studies suggested the interactions between the C-13 sidechain of docetaxel and additional binding sites shown in FIG. 18(C) may provide docetaxel with a tighter and more stable binding capability compared to rutin and acarbose.

Although this invention is described in detail with reference to preferred embodiments thereof, these embodiments are offered to illustrate but not to limit the invention. It is possible to make other embodiments that employ the principles of the invention and that fall within its spirit and scope as defined by the claims appended hereto.

The contents of all documents and references cited herein are hereby incorporated by reference in their entirety.

What is claimed is:

1. A method for preventing or treating an atherosclerosis-related disorder in a subject in need thereof, comprising administering to the subject an effective amount of a desmocollin 1 (DSC1) inhibitor, such that the atherosclerosis-related disorder is prevented or treated, wherein the DSC1 inhibitor comprises a low molecular weight compound set forth in Table 2, or a pharmaceutically acceptable salt or biologically active derivative thereof; a peptide comprising a fragment of apoA-I that inhibits DSC1-apoA-I interactions; an anti-DSC antibody specific for DSC1; or an antisense oligonucleotide or a small interfering RNA that targets DSC1 mRNA and/or inhibits DSC1 expression.

2. The method of claim 1, wherein the DSC1 inhibitor promotes HDL biogenesis in the subject.

3. The method of claim 1, wherein the DSC1 inhibitor is an inhibitor of: DSC1 expression; DSC1 binding to apoA-I protein; and/or DSC1 biological activity.

4. (canceled)

5. The method of claim 1, wherein the DSC1 inhibitor binds amino acid residues 130-218 of mature DSC1 (the EC2 repeat) and/or amino acid residues 442-538 of mature DSC1 (the EC5 repeat plus the region between the EC4 and EC5 repeats).

6.-7. (canceled)

8. The method of claim 1, wherein the DSC1 inhibitor is acarbose, rutin, or docetaxel, or a pharmaceutically acceptable salt or biologically active derivative thereof.

9. (canceled)

10. The method of claim 1, wherein the anti-DSC antibody is specific for amino acid residues 442-538 of DSC1.

11. The method of claim 10, wherein the anti-DSC antibody is a polyclonal antibody or a monoclonal antibody.

12. (canceled)

13. The method of claim 1, wherein the atherosclerosis-related disorder is atherosclerosis, atherosclerotic cardiovascular disease (ASCVD), or another high-density lipoprotein (HDL) biogenesis-linked disease, disorder or condition.

14. The method of claim 1, wherein the subject suffers from, or is at risk of, HDL deficiency, a lysosomal storage disease, Tangier disease, Niemann-Pick disease type A, Niemann-Pick disease type B, or Niemann-Pick disease type C.

15.-23. (canceled)

24. A method for promoting HDL biogenesis in a subject in need thereof, comprising administering to the subject an effective amount of a desmocollin 1 (DSC1) inhibitor, such that HDL biogenesis is promoted in the subject, wherein the DSC1 inhibitor comprises a low molecular weight compound set forth in Table 2, or a pharmaceutically acceptable salt or biologically active derivative thereof; a peptide comprising a fragment of apoA-I that inhibits DSC1-apoA-I interactions; an anti-DSC antibody specific for DSC1; or an antisense oligonucleotide or a small interfering RNA that targets DSC1 mRNA and/or inhibits DSC1 expression.

25. The method of claim 24, wherein the DSC1 inhibitor is an inhibitor of: DSC1 expression; DSC1 binding to apoA-I protein; and/or DSC1 biological activity.

26. (canceled)

27. The method of claim 24, wherein the DSC1 inhibitor binds amino acid residues 442-538 of DSC1.

28.-31. (canceled)

32. The method of claim 24, wherein the anti-DSC antibody is specific for amino acid residues 442-538 of DSC1.

33. The method of claim 32, wherein the anti-DSC antibody is a polyclonal antibody or a monoclonal antibody.

34.-36. (canceled)

37. The method of claim 24, wherein the subject suffers from atherosclerosis, atherosclerotic cardiovascular disease (ASCVD), HDL deficiency, a lysosomal storage disease, Tangier disease, Niemann-Pick disease type A, Niemann-Pick disease type B, or Niemann-Pick disease type C.

38.-50. (canceled)

51. A pharmaceutical composition comprising a desmocollin 1 (DSC1) inhibitor and a pharmaceutically acceptable diluent, carrier, or excipient, wherein the DSC1 inhibitor comprises a low molecular weight compound set forth in Table 2, or a pharmaceutically acceptable salt or biologically active derivative thereof; a peptide comprising a fragment of apoA-I that inhibits DSC1-apoA-I interactions; an anti-DSC antibody specific for DSC1; or an antisense oligonucleotide or a small interfering RNA that targets DSC1 mRNA and/or inhibits DSC1 expression.

52. (canceled)

53. The pharmaceutical composition of claim 51, wherein the DSC1 inhibitor specifically binds amino acid residues 442-538 of DSC1.

54.-56. (canceled)

57. A method for diagnosing an atherosclerosis-related disorder or a high-density lipoprotein (HDL) biogenesis-linked disease, disorder or condition in a subject comprising:
a) obtaining a biological sample from the subject;
b) detecting an expression level of DSC1 in the biological sample using an anti-DSC1 antibody or a nucleic acid specific for DSC1 RNA;
c) diagnosing the subject as having an atherosclerosis-related disorder or a high-density lipoprotein (HDL) biogenesis-linked disease, disorder or condition, or having a predisposition therefor, or being at risk therefor, when the expression level of DSC1 in the biological sample from the subject is higher than the expression level of DSC1 in a control biological sample from a control subject.

58. The method of claim 57, further comprising detecting an expression level of a biomarker for atherosclerotic disease in the biological sample, and diagnosing the subject as having an atherosclerosis-related disorder or a high-density lipoprotein (HDL) biogenesis-linked disease, disorder or condition or a predisposition therefor, or being at risk therefor, when the expression level of DSC1 in the biological sample from the subject is higher than the expression level of DSC1 in a control biological sample from a control subject and when the expression level of the biomarker is higher or lower than the expression level of the biomarker in the control biological sample.

59.-60. (canceled)

61. The method of claim 57, wherein the biological sample comprises whole blood, plasma or serum.

62. The method of claim 58, wherein the biomarker is an inflammatory biomarker, a biomarker of endothelial cell, platelet and leukocyte damage, activation, and adhesion, or a biomarker of macrophage monocytes.
